(19) United States
(12) Patent Application Publication
(10) Pub. No.: US 2025/0376446 A1
(43) Pub. Date: Dec. 11, 2025

(54) CRYSTALINE FORMS OF A SYNTHETIC CANNABINOID

(71) Applicant: Jazz Pharmaceuticals Research UK Limited, Sittingbourne (GB)

(72) Inventors: Hannah E. STRAKER, Sittingbourne (GB); Lynn MCMILLAN, Sittingbourne (GB); Keith LORIMER, Craigavon (IE); Catherine GREENAN, Craigavon (IE)

(21) Appl. No.: 18/874,175

(22) PCT Filed: Jun. 15, 2023
(86) PCT No.: PCT/GB2023/051563

§ 371 Date: Dec. 12, 2024

(30) Foreign Application Priority Data
Jun. 15, 2022 (GB) 2208810.8

(51) Int. Cl. C07D 231/12; A61K 31/415; A61P 25/08

(52) U.S. Cl. CPC C07D 231/12; A61K 31/415; A61P 25/08

(57) ABSTRACT

The present invention relates to crystalline forms of the synthetic, non-natural cannabinoid (1′R,2′R)-5′-methyl-4-(1-methyl-1H-pyrazol-4-yl)-2′-(prop-1-en-2-yl)-1′,2′,3′,4′-tetrahydro-[1,1′-biphenyl]-2,6-diol, methods for their production, and their use in therapy. The crystalline forms are easier to handle than other solid forms, and are useful in the manufacture of medicaments for the treatment of conditions associated with seizure.

### RELATED APPLICATIONS

The present application is related to, and claims the benefit of, GB 2208810.8 filed on 15 Jun. 2022 (15.06.2022), the contents of which are hereby incorporated by reference in their entirety.

### FIELD OF THE INVENTION

The present invention relates to crystalline forms of a synthetic cannabinoid, methods for their production, and their use in therapy.

### BACKGROUND TO THE INVENTION

Cannabinoids are natural and synthetic compounds structurally or pharmacologically related to the constituents of the cannabis plant or to the endogenous agonists (endocannabinoids) of the cannabinoid receptors CB1 or CB2. The only way in nature in which these compounds are produced is by the cannabis plant. Cannabis is a genus of flowering plants in the family Cannabaceae, comprising the species Cannabis sativa, Cannabis indica, and Cannabis ruderalis (sometimes considered as part of Cannabis sativa).

Cannabis plants comprise a highly complex mixture of compounds. At least 568 unique molecules have been identified in cannabis extracts. Among these compounds are cannabinoids, terpenoids, sugars, fatty acids, flavonoids, other hydrocarbons, nitrogenous compounds, and amino acids.

Cannabinoids exert their physiological effects through a variety of receptors including, but not limited to, adrenergic receptors, cannabinoid receptors (CB1 and CB2), GPR55, GPR3, or GPR5. The principal cannabinoids present in cannabis plants are cannabinoid acids Δ9-tetrahydrocannabinolic acid (Δ9-THCA) and cannabidiolic acid (CBDA) with small amounts of their respective neutral (decarboxylated) cannabinoids. In addition, cannabis may contain lower levels of other minor cannabinoids.

There are currently four cannabinoid-based pharmaceutical approved products on the market. These are: dronabinol (Marinol®) which is a synthetic tetrahydrocannabinol (THC) approved for the treatment of loss of appetite in AIDS and the treatment of severe nausea and vomiting caused by cancer chemotherapy; nabilone (Cesamet®) which is a synthetic cannabinoid and an analogue of THC which is approved for the treatment of nausea and vomiting caused by cytotoxic chemotherapy unresponsive to conventional antiemetics; nabiximols (Sativex®) a mixture of two cannabis plant extracts approved for the treatment of neuropathic pain, spasticity, overactive bladder, and other symptoms of multiple sclerosis; and highly purified botanical CBD (Epidiolex®) approved in the United States for the treatment of Dravet syndrome and Lennox-Gastaut syndrome in children and adults over the age of 2 years.

Over 100 different cannabinoids have been identified. These cannabinoids can be split into different groups as follows: phytocannabinoids; endocannabinoids and synthetic cannabinoids (which may be novel cannabinoids or synthetically produced versions of phytocannabinoids or endocannabinoids). The Handbook of Cannabis (Pertwee, 2014) provides an overview of known cannabinoids.

Cannabidiol (CBD) is a major cannabinoid constituent of Cannabis species, such as the hemp plant (Cannabis sativa). Unlike other cannabinoids, such as THC, cannabidiol does not bind to CB1 or CB2 receptors, or its binding to the receptors is negligible in terms of inducing a pharmacological effect. Thus, cannabidiol does not cause the central or peripheral nervous system effects mediated by the CB1 or CB2 receptors. CBD has little or no psychotropic (cannabimimetic) activity and its molecular structure and properties are substantially different from those of other cannabinoids.

Extracted cannabidiol is an amorphous or semi-crystalline semi-solid. Due to the highly aliphatic nature of cannabidiol, which contains few polarisable groups, cannabidiol is difficult to solubilise. The high LogP of cannabidiol means that it is challenging to prepare a suitable pharmaceutical product. Oral formulations of cannabidiol are particularly difficult to prepare, and often have poor bioavailability. Highly lipophilic drugs including CBD also precipitate in the GI tract leading to higher elimination and poorer absorption (O'Sullivan). Thus, there is a need for alternatives to cannabidiol which are crystalline, which can be more easily formulated into a pharmaceutical product, and which have improved bioavailability.

The present invention has been devised in light of these considerations.

### BRIEF SUMMARY OF THE INVENTION

At its most general, the present invention relates to crystalline forms of the synthetic cannabidiol-like cannabinoid (1′R,2′R)-5′-methyl-4-(1-methyl-1H-pyrazol-4-yl)-2′-(prop-1-en-2-yl)-1′,2′,3′,4′-tetrahydro-[1,1′-biphenyl]-2,6-diol:

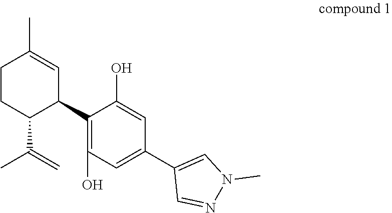 [SMILES: C=C(C)[C@@H]1CCC(C)=C[C@H]1c1c(O)cc(-c2cnn(C)c2)cc1O]

In a first aspect of the invention there is provided a crystalline Form B of compound 1. The inventors have found that Form B has excellent chemical stability. For example, Form B is particularly stable to various stress conditions including compression and milling, which aids processing and handling during formulation of a pharmaceutical product. In addition, Form B displays excellent stability on storage under high humidity conditions and at elevated temperatures. Thus, a pharmaceutical product comprising Form B would not require cold storage or a low temperature supply chain. Form B also has a high melting-point, further improving processability.

Form B may be characterised by an X-ray powder diffraction (XRPD) pattern comprising a peak at 8.33 and 23.16±0.2 (°2θ).

Form B may be further characterised by a thermogravimetric differential thermal analysis (TG/DTA) thermogram comprising a peak with an onset temperature (Tₒₙₛₑₜ) of 195±2° C. Form B may be further characterised by an XRPD pattern further comprising peaks at 12.61, 15.65, 16.88, 20.14, 23.62, 23.99 and 26.44±0.2 (°2θ). Form B may be further characterised by an XRPD pattern further comprising peaks at 12.45, 13.74, 16.70,17.75, 19.91, 20.88, 22.06, 25.06 and 25.85±0.2 (°2θ). Form B may be further characterised by an XRPD pattern further comprising peaks at 13.96, 19.73, 21.91 and 24.66±0.2 (°2θ). Form B may have an XRPD pattern substantial as shown in FIG. 6. Form B may be anhydrous.

In a second aspect of the invention, there is provided a crystalline Form A of compound 1.

Form A may be characterised by an XRPD pattern comprising a peak at 14.92±0.2(°2θ).

Form A may be further characterised an XRPD pattern comprising peaks at 12.91,20.20, 24.03, 24.16 and 27.54±0.2 (°2θ). Form A may be further characterised by a TG/DTA thermogram comprising a peak with an onset temperature (Tₒₙₛₑₜ) of 179±2° C. Form A may be further characterised by an XRPD pattern further comprising peaks at 12.21, 17.89, 19.15 and 25.12±0.2 (°2θ). Form A may be further characterised by an XRPD pattern further comprising peaks at 24.53±0.2 (°2θ). Form A may be anhydrous.

In a third aspect of the invention, there is provided a crystalline Form C of compound 1.

Form C may be characterised by an XRPD pattern comprising peaks at 4.51,13.86, 16.39, 18.57, 20.94, 22.25 and 25.66+0.2 (°2θ).

Form C may be further characterised by a TG/DTA thermogram comprising a peak with a Tₒₙₛₑₜ of 76±2° C. Optionally, the peak is associated with a weight loss of from 5 wt % to 18 wt %. Form C may be an ethanol solvate.

In a fourth aspect of the invention, there is provided a crystalline Form E of compound 1.

Form E may be characterised by an XRPD pattern comprising peaks at 9.02,13.89, 16.41, 18.60, 22.02, 22.29 and 25.69±0.2 (°2θ).

Form E may be further characterised by an XRPD pattern further comprising peaks at 11.48 and 19.54±0.2 (°2θ).

In a fifth aspect of the invention, there is provided a composition comprising compound 1, wherein 5 wt % or more, such as 50 wt % or more, such as 90 wt % or more, such as 95% wt or more of the compound 1 is in the crystalline form of any of the first, second, third or fourth aspects.

In a sixth aspect of the invention, there is provided a pharmaceutical composition comprising the crystalline form of any of the first, second, third or fourth aspects, or the composition the fifth aspect, together with one or more ingredients selected from carriers, diluents, excipients, adjuvants, fillers, buffers, binders, disintegrants, preservatives, antioxidants, lubricants, stabilisers, solubilisers, surfactants, masking agents, colouring agents, flavouring agents, and sweetening agents.

In one embodiment, the pharmaceutical composition is in a form selected from a tablet, a granule, a powder, a lozenge, a pastilles, a capsules or a pill.

In a seventh aspect of the invention, there is provided the crystalline form of any of the first, second, third or fourth aspects; the composition the fifth aspect; or the pharmaceutical composition of the sixth aspect, for use in a method of treatment.

In one embodiment, the method of treatment is a method of treating epilepsy.

In one embodiment, the method of treatment is a method of treating seizures, such as generalised seizures, focal-onset seizures or tonic-clonic seizures.

In a ninth aspect of the invention, there is provided a method for preparing a crystalline form of compound 1, the method comprising the steps of:
a) providing a composition comprising compound 1 and an organic solvent;
b) agitating the composition; and
c) isolating the solids.

In a tenth aspect of the invention, there is provided a method for preparing a crystalline form of compound 1, the method comprising the steps of:
a) Providing a composition comprising compound 1 and a first solvent;
b) Adding a second solvent; and
c) Isolating the solids.

These and other aspects and embodiments of the invention are described in further detail below.

### BRIEF SUMMARY OF THE DRAWINGS

There present invention is described with reference to the figures listed below:

FIG. 1 is an XRPD spectrum of amorphous compound 1 prepared by melt quench.

FIG. 2 is a cyclic hyper DSC thermogram for compound 1 from −50 to 250° C. at 300° C. per minute.

FIG. 3 is an XRPD spectrum of compound 1 Form A.

FIG. 4 is a TG/DTA thermogram for compound 1 Form A analysed from 30 to 300° C. at 10° C. per minute.

FIG. 5 is a DSC thermogram for compound 1 Form A analysed from 30 to 300° C. at 10° C. per minute.

FIG. 6 is an XRPD spectrum of compound 1 Form B.

FIG. 7 is a TG/DTA thermogram for compound 1 Form B.

FIG. 8 shows the XRPD patterns of Form B material before (top) and after (bottom) milling.

FIG. 9 shows the XRPD patterns of Form B material before (top) and after (bottom) compression.

FIG. 10 is an XRPD spectrum for compound 1 Form C.

FIG. 11 is a TG-DTA thermogram for compound 1 Form C.

FIG. 12 is an XRPD spectrum for compound 1 Form E.

FIG. 13A shows the XRPD patterns of Form C material (top) and Form E material (bottom).

FIG. 13B shows an expanded view of the XRPD patterns of Form C material (top) and Form E material (bottom) between 12 and 16 °2θ.

FIG. 14 shows the evaluation of compound 1 in the MEST test in the mouse (*** indicates a P<0.001 significant change in threshold when compared to own vehicle).

### DETAILED DESCRIPTION OF THE INVENTION

The present invention provides crystalline forms of the synthetic cannabinoid compound 1.

Characterisation of a crystalline material, such as a crystalline form of compound 1, and differentiation of a crystalline material from an amorphous material can be achieved using known techniques. In particular, verification of the form can be achieved using techniques such as melting point, differential scanning calorimetry, thermogravimetric differential thermal analysis, optical microscopy and X-ray powder diffraction.

### Form A

In one aspect, the present invention provides a crystalline Form A of compound 1.

Crystalline Form A has a high melting point (above 150° C.), which is useful for storage, preparation and handling.

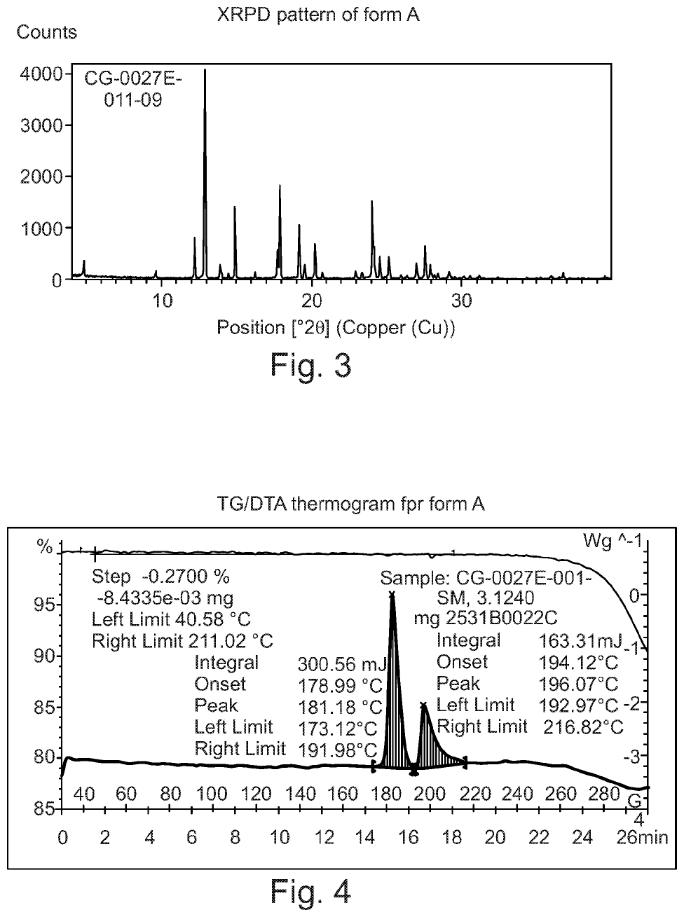

The XRPD pattern of crystalline Form A displays characteristic peaks (see, e.g., FIG. 3). The XRPD pattern of the material may be determined using standard techniques, such as using a Panalytical Xpert Pro diffractometer equipped with a Cu X-ray tube and a Pixcel detector system or a Panalytical Empyrean diffractometer equipped with a Cu X-ray tube and a PIXcel 1D-Medipix3 detector system. Sample analysis may be performed at, for example, ambient temperature in transmission mode, with a spin rate of 60 rpm. Characteristic peaks may be identified based on their position (°2θ). Characteristic peaks may be further identified by their relative intensity in comparison to the largest identified peak using a suitable cut-off value (for example, a relative intensity of greater than 10%).

In one embodiment, the XRPD pattern of the Form A compound exhibits a characteristic peak at 14.92±0.2 (°2θ), such as measured using Cu-Kα irradiation (1.54060 Å). This characteristic peak is only observed in Form A and has not been observed in any other identified form of compound 1.

In one embodiment, Form A is characterised by an XRPD pattern comprising at least one peak at a position (°2θ) selected from 12.91, 20.20, 24.03, 24.16 and 27.54±0.2,such as measured using Cu-K_{α} irradiation (1.54060 Å). This pattern of characteristic peaks is only observed in Form A and has not been observed in any other identified form of compound 1.

Preferably, Form A is characterised by an XRPD pattern comprising at least two peaks, more preferably at least three peaks, even more preferably at least four peaks, and most preferably at least five peaks at positions (°2θ) selected from 12.91, 20.20, 24.03, 24.16 and 27.54±0.2, such as measured using Cu-K_{α} irradiation (1.54060 Å).

In one embodiment, Form A is characterised by an XRPD pattern comprising peaks at 12.91, 20.20, 24.03, 24.16 and 27.54±0.2, such as measured using Cu-K_{α} irradiation (1.54060 Å).

In one embodiment, Form A is characterised by an XRPD pattern further comprising peaks at 12.21, 17.89, 19.15 and 25.12±0.2, such as measured using Cu-K_{α} irradiation (1.54060 Å).

In one embodiment, Form A is characterised by an XRPD pattern further comprising a peak at 24.53±0.2, such as measured using Cu-K_{α} irradiation (1.54060 Å).

In one embodiment, Form A has an XRPD pattern substantial as shown in FIG. 3.

The TG/DTA thermogram of crystalline Form A displays characteristic peaks (FIG. 4). The TG/DTA thermogram may be determined using standard techniques, such as using a Mettler Toledo TGA/DSC1 STARe using iridium and tin as calibration standards and with a heating rate of 10° C./minute to a maximum of 300° C.

In one embodiment, Form A is characterised by a TG/DTA thermogram comprising a peak with an onset temperature (Tₒₙₛₑₜ) of 179±2° C., such as measured at a heating rate of 10° C./min. Typically, the onset temperature is associated with a minimal weight loss, for example of 10 wt % or less, such as 5 wt % or less, or 2 wt % or less. Without wishing to be bound by theory, the inventors attribute this peak to the melt of Form A.

In one embodiment, Form A has a TG/DTA thermogram substantial as shown in FIG. 4.

Phase transitions may also be seen in the DSC thermogram. The DSC thermogram may be determined using standard techniques, such using a Perkin Elmer DSC8500 Differential Scanning calorimeter with a heating rate of 10° C./minute to a maximum of 250° C.

In one embodiment, Form A is anhydrous (does not contain bound water). In a preferred embodiment the Form A does not contain any bound solvent.

In one aspect, the invention provides a composition comprising compound 1, wherein 5 wt % or more of the compound 1 is Form A. In a preferred embodiment, the invention provides a composition comprising compound 1, wherein 10 wt % or more, more preferably 50 wt % or more, of the compound 1 is Form A.

In another aspect, the invention provides a composition comprising compound 1, wherein 85 wt % or more of the compound 1 is Form A. In a preferred embodiment, the invention provides a composition comprising compound 1, wherein 90 wt % or more, more preferably 95 wt % or more, even more preferably 98 wt % or more, of the compound 1 is Form A.

In one aspect, the invention provides a method for preparing crystalline Form A compound 1, the method comprising the steps of:
(a) providing a composition comprising compound 1 and a hydrocarbon solvent;
(b) sonicating the composition; and
(c) isolating the solids.

The compound 1 used in the preparation step (step a) may be any form of compound. Typically, compound 1 used in the preparation method is or comprises amorphous compound 1.

The hydrocarbon solvent may be an aliphatic or aromatic hydrocarbon solvent.

Examples of suitable aliphatic hydrocarbon solvents include linear alkanes such as pentane, hexane, heptane and octane; cycloalkanes such as cyclopentane, cyclohexane, cycloheptane and cyclooctane; and petroleum fractions such as kerosene and petroleum ether. Examples of suitable aromatic hydrocarbon solvents include benzene, toluene and xylene.

In one embodiment, the organic solvent is an aliphatic hydrocarbon solvent, preferably the organic solvent is heptane, hexane or pentane, more preferably the organic solvent is heptane.

The sonication step (step b) may be carried out intermittently, such as in 30 second intervals. Typically, the sonication step is carried out using a sonication intensity of from 100 W to 160 W, such as from 120 W to 140 W, such as about 130 W.

The isolation step (step c) may comprise isolating the solid material by any suitable method. Suitable methods include filtration and centrifugation.

In one aspect, the invention provides a crystalline form of compound 1 prepared by the method outlined in paragraphs [0053] to [0059].

### Form B

In one aspect, the present invention provides crystalline Form B of compound 1. Crystalline Form B material is particularly preferred because it has excellent chemical stability. Form B displays good stability to compression and milling, which aids processing and handling during formulation of a pharmaceutical product. Form B displays excellent stability on storage under high humidity conditions and at elevated temperatures. The indicates that a pharmaceutical product comprising Form B would not require cold storage or a low temperature supply chain. Form B also has a high melting-point, further improving processability.

The XRPD pattern of crystalline Form B displays characteristic peaks (see, e.g., FIG. 6). The XRPD pattern of the material may be determined using standard techniques (e.g., as set out for Form A, above).

In one embodiment, the XRPD pattern of the Form B compound exhibits characteristic peaks at 8.33 and 23.16±0.2 (°2θ), such as measured using Cu-Kα irradiation (1.54060 Å). These characteristic peaks are only observed in Form B and have not been observed in any other identified form of compound 1.

In one embodiment, Form B is characterised by an XRPD pattern comprising at least one peak at a position (°2θ) selected from 12.61, 15.65, 16.88, 20.14, 23.62, 23.99 and 26.44±0.2, such as measured using Cu-K_{α} irradiation (1.54060 Å). This pattern of characteristic peaks is only observed in Form B and has not been observed in any other identified form of compound 1.

Preferably, Form B is characterised by an XRPD pattern comprising at least two peaks, more preferably at least three peaks, even more preferably at least four peaks, and most preferably at least five peaks at positions (°2θ) selected from 12.61, 15.65, 16.88,20.14, 23.62, 23.99 and 26.44±0.2, such as measured using Cu-K_{α} irradiation (1.54060 Å).

In one embodiment, Form B is characterised by an XRPD pattern comprising peaks at 12.61, 15.65, 16.88, 20.14, 23.62, 23.99 and 26.44±0.2, such as measured using Cu-K_{α} irradiation (1.54060 Å).

In one embodiment, Form B is characterised by an XRPD pattern further comprising peaks at 12.45, 13.74, 16.70, 19.91, 20.88, 22.06, 25.06 and 25.85±0.2, such as measured using Cu-K_{α} irradiation (1.54060 Å).

In one embodiment, Form B is characterised by an XRPD pattern further comprising peaks at 13.96, 17.75, 19.73, 21.91 and 24.66±0.2, such as measured using Cu-K_{α} irradiation (1.54060 Å).

In one embodiment, Form B has an XRPD pattern substantial as shown in FIG. 6.

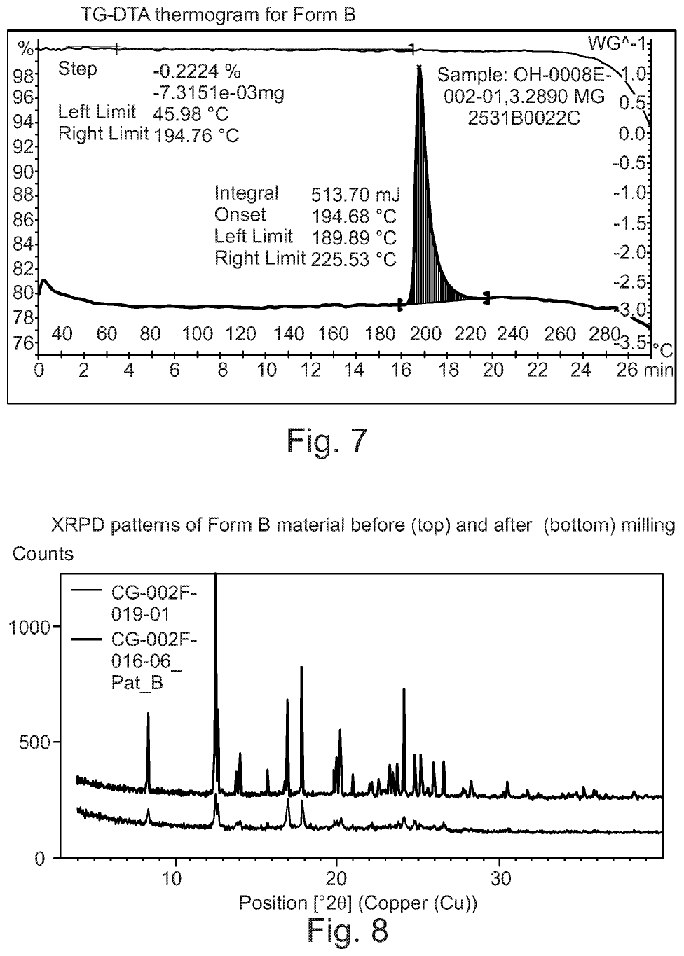

The TG/DTA thermogram of crystalline Form B displays characteristic peaks (FIG. 7). The TG/DTA thermogram may be determined using standard techniques (e.g. as set out for Form A, above).

In one embodiment, Form B is characterised by a TG/DTA thermogram comprising a peak with an onset temperature (Tₒₙₛₑₜ) of 195±2° C., such as measured at a heating rate of 10° C./min. Typically, the onset temperature is associated with a minimal weight loss, for example of 10 wt % or less, such as 5 wt % or less, or 2 wt % or less. Without wishing to be bound by theory, the inventors attribute this peak to the melt of Form B.

In one embodiment, Form B has a TG/DTA thermogram substantial as shown in FIG. 7.

In one embodiment, Form B is anhydrous (does not contain bound water). In a preferred embodiment the Form B does not contain any bound solvent.

In one aspect, the invention provides a composition comprising compound 1, wherein 5 wt % or more of the compound 1 is Form B. In a preferred embodiment, the invention provides a composition comprising compound 1, wherein 10 wt % or more, more preferably 50 wt % or more, of the compound 1 is Form B.

In another aspect, the invention provides a composition comprising compound 1, wherein 85 wt % or more of the compound 1 is Form B. In a preferred embodiment, the invention provides a composition comprising compound 1, wherein 90 wt % or more, more preferably 95 wt % or more, even more preferably 98 wt % or more, of the compound 1 is Form B.

In one aspect, the invention provides a method for preparing crystalline Form B compound 1, the method comprising the steps of:
(a) providing a composition comprising compound 1 and an organic solvent;
(b) agitating the composition; and
(c) isolating the solids.

The compound 1 used in the preparation step (step a) may be any form of compound. Typically, compound 1 used in the preparation method is or comprises amorphous compound 1.

The slurrying step (step b) may be carried out using mechanical agitation, such as stirring.

The organic solvent may be selected from linear alkanes such as pentane, hexane, heptane and octane; cycloalkanes such as cyclopentane, cyclohexane, methylcyclohexane, cycloheptane and cyclooctane; petroleum fractions such as kerosene and petroleum ether; aromatic hydrocarbon solvents such as benzene, toluene and xylene; esters such as ethyl acetate and isopropyl acetate; and ethers such as dioxane, tetrahydrofuran, 2-methyl tetrahydrofuran, methyl tert-butyl ether and cyclopentyl methyl ether.

The slurrying step (step b) may be carried out at any suitable temperature. Typically, the slurrying step is carried out at from 0° C. to 70° C., such as from 10° C. to 60° C.

In one embodiment, the slurrying step (step b) is carried out at from 10° C. to 30° C., such as from 15° C. to 25° C., such as about 20° C.

In another embodiment, the slurrying step (step b) is carried out at from 40° C. to 60° C., such as from 45°° C. to 55° C., such as about 50° C.

The isolation step (step c) may comprise isolating the solid material by any suitable method. Suitable methods include filtration and centrifugation.

In another aspect, the invention provides a method for preparing crystalline Form B compound 1, the method comprising the steps of:
a) providing a composition comprising compound 1 and an organic solvent;
b) heating the composition;
c) seeding the composition with crystalline Form B compound 1;
d) cooling the composition; and
e) isolating the solids.

Preferences for the preparation step (step a) and isolation step (step e) are set out above.

The heating step (step b) may be carried out to a predetermined maximum temperature. Typically, the predetermined maximum temperature is the lower of i) 10° C. lower than the solvent boiling point, preferably 5° C. or 3° C. lower than the solvent boiling point; or ii) 100° C.

The heating step (step b) may be carried out at a predetermined heating rate. Typically, the predetermined heating rate is from 0.2° C./min to 20° C./min, preferably from 0.2° C./min to 10° C./min, or 0.5° C./min to 5° C./min.

The cooling step (step d) may be carried out to a predetermined minimum temperature. Typically, the predetermined minimum temperature is ambient temperature (˜20° C.).

The cooling step (step d) may be carried out at a predetermined cooling rate. Typically, the predetermined heating rate is from 0.05° C./min to 10° C./min, preferably from 0.05° C./min to 5° C./min, or 0.1° C./min to 2° C./min.

In another aspect, the invention provides a method for preparing crystalline Form B compound 1, the method comprising the steps of:
a) providing a composition comprising compound 1 and a first solvent;
b) adding a second solvent; and
c) isolating the solids.

The compound 1 used in the preparation step (step a) may be any form of compound. Typically, compound 1 used in the preparation method is or comprises amorphous compound 1.

The first solvent may be selected from esters such as ethyl acetate and isopropyl acetate; ethers such as dioxane, tetrahydrofuran, 2-methyl tetrahydrofuran, methyl tert-butyl ether and cyclopentyl methyl ether; ketones such as acetone; and nitriles such as acetonitrile. In a preferred embodiment, ethyl acetate is used.

The preparation step (step a) may be carried out at any suitable temperature. Typically the preparation step is carried out at 0° C. to 80° C., such as from 20° C. to 80° C.

In a preferred embodiment, the preparation step (step a) is carried out at from 50° C. to 80° C., more preferably from 65° C. to 75° C.

In a preferred embodiment, the method comprises a cooling step (step a-1) between the preparation step (step a) and the addition step (step b). The cooling step may be carried out at a predetermined cooling rate. Typically, the predetermined heating rate is from 0.05° C./min to 10° C./min, preferably from 0.05° C./min to 5° C./min, more preferably from 0.1° C./min to 2° C./min, and even more preferably from 0.1° C./min to 1° C./min.

The second solvent may be selected from linear alkanes such as pentane, hexane, heptane and octane; cycloalkanes such as cyclopentane, cyclohexane, methylcyclohexane, cycloheptane and cyclooctane; petroleum fractions such as kerosene and petroleum ether; and aromatic hydrocarbon solvents such as benzene, toluene and xylene. In a preferred embodiment heptane is used.

The addition step (step b) may be carried out at any suitable temperature. Typically, the addition step is carried out at from 0° C. to 70° C., such as from 10° C. to 60° C.

In a preferred embodiment, the addition step (step b) is carried out at from 10° C. to 30° C., more preferably from 15° C. to 25° C., even more preferably from 20° C. to 25° C., such as about 20° C.

In a preferred embodiment, the method comprises an aging step (step b-1) between the addition step (step b) and the isolation step (step c). The aging step (step b-1) comprises:
b-1) holding the crystallisation mixture (the product of step b) at a predetermined temperature for a predetermined length of time.

Typically, the predetermined temperature is from 10° C. to 30° C., such as from 15° C. to 25° C., or from 20° C. to 25° C., such as about 20° C. Typically, the predetermined length of time is from 1 hour to 24 hours, such as from 2 hours to 12 hours, or from 4 hours to 8 hours.

In one embodiment, the aging step (step b-1) comprises stirring the crystallisation mixture at a predetermined temperature for a predetermined length of time.

Preferences for the isolation step (step c) are set out above.

In one aspect, the invention provides a crystalline form of compound 1 prepared by the method outlined in paragraphs [0076] to [0083], [0084] to [0089] or [0090] to [0102].

### Form C

In one aspect, the present invention provides crystalline Form C of compound 1.

The XRPD pattern of crystalline Form C displays characteristic peaks (see, e.g., FIG. 10). The XRPD pattern of the material may be determined using standard techniques (e.g., as set out for Form A, above).

In one embodiment, Form C is characterised by an XRPD pattern comprising at least one peak at a position (°2θ) selected from 4.51, 13.86, 16.39, 18.57, 20.94, 22.25 and 25.66±0.2, such as measured using Cu-K_{α} irradiation (1.54060 Å). This pattern of characteristic peaks is only observed in Form C and has not been observed in any other identified form of compound 1.

Preferably, Form C is characterised by an XRPD pattern comprising at least two peaks, more preferably at least three peaks, even more preferably at least four peaks, and most preferably at least five peaks at positions (°2θ) selected from 4.51, 13.86, 16.39, 18.57, 20.94, 22.25 and 25.66±0.2, such as measured using Cu-K_{α} irradiation (1.54060 Å).

In one embodiment, Form C is characterised by an XRPD pattern comprising peaks at 4.51, 13.86, 16.39, 18.57, 20.94, 22.25 and 25.66±0.2, such as measured using Cu-K_{α} irradiation (1.54060 Å).

In one embodiment, Form C is characterised by an XRPD pattern further comprising peaks at 11.45, 17.86, 19.51 and 21.98±0.2, such as measured using Cu-K_{α} irradiation (1.54060 Å).

In one embodiment, Form C has an XRPD pattern substantial as shown in FIG. 10.

The TG/DTA thermogram of crystalline Form C displays characteristic peaks (FIG. 11). The TG/DTA thermogram may be determined using standard techniques (e.g. as set out for Form A, above).

In one embodiment, Form C is characterised by a TG/DTA thermogram comprising a broad peak with an onset temperature (Tₒₙₛₑₜ) of 76±2° C., such as measured at a heating rate of 10° C./min. Typically, the peak is associated with a weight loss of from 5 wt % to 18 wt %, such as from 8 wt % to 15 wt %, such as about 11 wt %. Without wishing to be bound by theory, the inventors attribute this peak to the desolvation of Form C.

In one embodiment, Form C has a TG/DTA thermogram substantial as shown in FIG. 11.

In one embodiment, Form C is an ethanol solvate. In a preferred embodiment, Form C is an ethanol mono-solvate.

In one aspect, the invention provides a composition comprising compound 1, wherein 5 wt % or more of the compound 1 is Form C. In a preferred embodiment, the invention provides a composition comprising compound 1, wherein 10 wt % or more, more preferably 50 wt % or more, of the compound 1 is Form C.

In another aspect, the invention provides a composition comprising compound 1, wherein 85 wt % or more of the compound 1 is Form C. In a preferred embodiment, the invention provides a composition comprising compound 1, wherein 90 wt % or more, more preferably 95 wt % or more, even more preferably 98 wt % or more, of the compound 1 is Form C.

In one aspect, the invention provides a method for preparing crystalline Form C compound 1, the method comprising the steps of:
(a) providing a composition comprising compound 1 and a solvent comprising ethanol;
(b) sonicating the composition; and
(c) isolating the solids.

The compound 1 used in the preparation step (step a) may be any form of compound. Typically, compound 1 used in the preparation method is or comprises amorphous compound 1.

The solvent comprises ethanol. Typically, the solvent comprises ethanol in an amount of 80% v/v or more, preferably 85% v/v or more, more preferably 90% v/v or more and even more preferably 95% v/v or more. In some embodiments, the solvent comprises 96% v/v ethanol. In some embodiments, the solvent consists of ethanol.

Optionally, the solvent further comprises water. Typically, the solvent comprises water in an amount of 20% v/v or less, preferably 15% v/v or less, more preferably 10% v/v/ or less and even more preferably 5% v/v/ or less. In some embodiment, the solvent comprises 4% v/v water.

The sonication step (step b) may be carried out intermittently, such as in 30 second intervals. Typically, the sonication step is carried out using a sonication intensity of from 100 W to 160 W, such as from 120 W to 140 W, such as about 130 W.

The isolation step (step c) may comprise isolating the solid material by any suitable method. Suitable methods include filtration and centrifugation.

In one aspect, the invention provides a crystalline form of compound 1 prepared by the method outlined in paragraphs [0117] to [0122].

### Form E

In one aspect, the present invention provides crystalline Form E of compound 1.

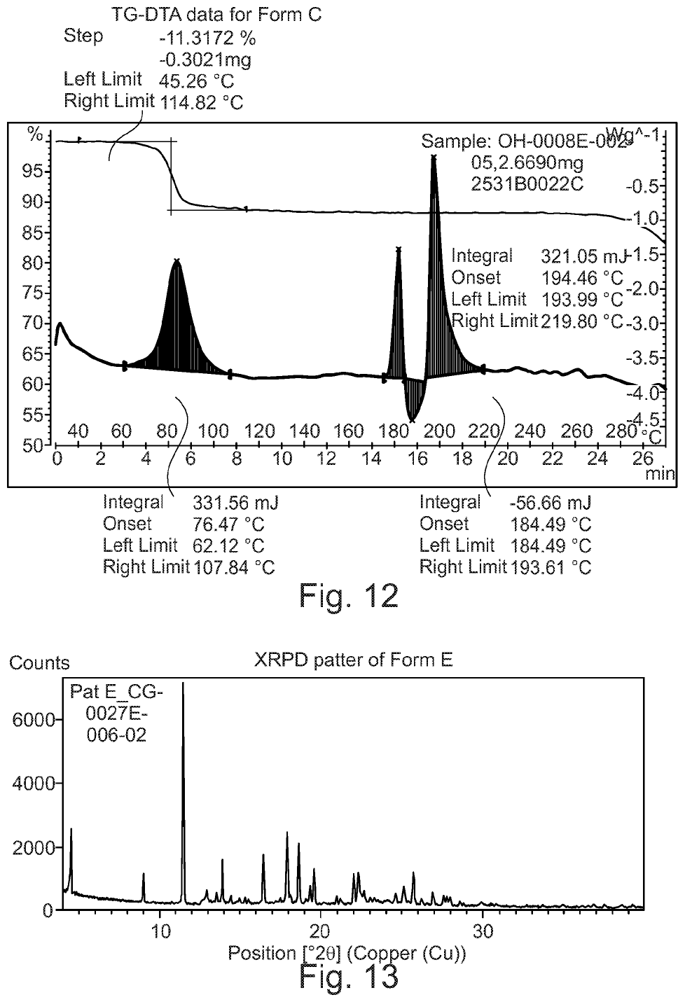

The XRPD pattern of crystalline Form E displays characteristic peaks (see, e.g., FIG. 12 The XRPD pattern of the material may be determined using standard techniques (e.g., as set out for Form A, above).

In one embodiment, Form E is characterised by an XRPD pattern comprising at least one peak at a position (°2θ) selected from 9.02, 13.89, 16.41, 17.89, 18.60, 22.02, 22.29 and 25.69±0.2, such as measured using Cu-K_{α} irradiation (1.54060 Å). This pattern of characteristic peaks is only observed in Form E and has not been observed in any other identified form of compound 1.

Preferably, Form E is characterised by an XRPD pattern comprising at least two peaks, more preferably at least three peaks, even more preferably at least four peaks, and most preferably at least five peaks at positions (°2θ) selected from 9.02, 13.89, 16.41, 17.89, 18.60, 22.02, 22.29 and 25.69±0.2, such as measured using Cu-K_{α} irradiation (1.54060 Å).

In one embodiment, Form E is characterised by an XRPD pattern comprising peaks at 9.02, 13.89, 16.41, 17.89, 18.60, 22.02, 22.29 and 25.69±0.2, such as measured using Cu-K_{α} irradiation (1.54060 Å).

In one embodiment, Form E is characterised by an XRPD pattern further comprising peaks at 11.48 and 19.54±0.2, such as measured using Cu-K_{α} irradiation (1.54060 Å).

In one embodiment, Form E has an XRPD pattern substantial as shown in FIG. 12.

In one embodiment, Form E is a solvate.

In one aspect, the invention provides a method for preparing crystalline Form E compound 1, the method comprising the steps of:
(a) providing a composition comprising compound 1 and a solvent comprising ethanol and methanol;
(b) agitating the composition; and
(c) isolating the solids.

The compound 1 used in the preparation step (step a) may be any form of compound. Typically, compound 1 used in the preparation method is or comprises amorphous compound 1.

The slurrying step (step b) may be carried out using mechanical agitation, such as stirring.

The solvent comprises ethanol. Typically, the solvent comprises ethanol in an amount of 80% v/v or more, preferably 85% v/v or more, more preferably 90% v/v or more and even more preferably 95% v/v or more. In some embodiments, the solvent comprises 95% v/v ethanol.

The solvent further comprises methanol. Typically, the solvent comprises methanol in an amount of 20% v/v or less, preferably 15% v/v or less, more preferably 10% v/v/or less and even more preferably 5% v/v/or less. In some embodiments, the solvent comprises 5% v/v methanol.

In some embodiment, the solvent is IMS (industrial methylated spirit).

In one embodiment, the slurrying step (step b) is carried out at from 10° C. to 30° C., such as from 15° C. to 25° C., such as about 20° C.

The isolation step (step c) may comprise isolating the solid material by any suitable method. Suitable methods include filtration and centrifugation.

In one aspect, the invention provides a crystalline form of compound 1 prepared by the method outlined in paragraphs [0132] to [0139].

### Pharmaceutical Compositions

While it is possible for the crystalline forms A, B, C and E to be administered alone, it is preferable to administer a pharmaceutical composition (e.g., a formulation, preparation, or medicament) comprising Form A, B, C or E together with one or more other pharmaceutically acceptable ingredients.

Accordingly, the invention provides a pharmaceutical composition comprising Form A, B, C or E, together with one or more pharmaceutically acceptable ingredients.

In preferred embodiments, the pharmaceutical composition comprises Form A or Form B.

In more preferred embodiment, the pharmaceutical composition comprises Form B.

Suitable pharmaceutically acceptable ingredients (e.g. carriers, diluents, excipients, etc.) can be found in standard pharmaceutical texts, for example, Remington: The Science and Practice of Pharmacy, 20th Edition, 2000, pub. Lippincott, Williams & Wilkins; and Handbook of Pharmaceutical Excipients, 9th edition, 2020, pub. Pharmaceutical Press.

Examples of suitable pharmaceutically acceptable ingredients include pharmaceutically acceptable carriers, diluents, excipients, adjuvants, fillers, buffers, binders, disintegrants, preservatives, antioxidants, lubricants, stabilisers, solubilisers, surfactants (e.g., wetting agents), masking agents, colouring agents, flavouring agents, and sweetening agents.

The pharmaceutical composition may be in any suitable form. Examples of suitable forms include tablets (including, e.g., coated tablets), granules, powders, lozenges, pastilles, capsules (including, e.g., hard and soft gelatin capsules), and pills.

### Medical Treatment

The inventors have found that compound 1 is biologically active. The worked examples demonstrate that compound 1 displays anticonvulsant activity in a mouse model of seizure. As such, crystalline forms of compound 1, as well as pharmaceutical compositions comprising crystalline forms of compound 1, will be useful in medical treatment.

Accordingly, the invention provides Form A, B, C or E, for use in a method of treatment, for example for use in a method of treatment of the human or animal body by therapy (i.e. a method of therapy).

The invention also provides Form A, B, C or E for use as a medicament.

The invention also provides a method of treatment comprising administering to a subject in need of treatment a therapeutically effective amount of Form A, B, C or E.

The invention also provides the use of Form A, B, C or E for the manufacture of a medicament.

The invention also provides use of Form A, B, C or E in a method of treatment.

### Conditions Treated

The inventors have found that the compound 1 displays anticonvulsant activity in a mouse model of generalised seizure. Accordingly, crystalline forms of compound 1, such as Form A, B, C or E, as well as pharmaceutical compositions comprising crystalline forms of compound 1, such as Form A, B, C or E, will be useful in the treatment of certain conditions associated with seizure.

Similarly, crystalline forms of compound 1, such as Form A, B, C or E, as well as pharmaceutical compositions comprising crystalline forms of compound 1, such as Form A, B, C or E, will be useful as medicaments for treating (and in the manufacture of medicaments for treating) certain conditions associated with seizure.

In a preferred embodiment, the condition associated with seizure is epilepsy.

In one embodiment, the condition associated with seizure is generalised-onset seizure, such as generalised-onset seizure associated with epilepsy.

In one embodiment, the condition associated with seizure is focal-onset seizures, such as focal-onset seizure associated with epilepsy.

In one embodiment, the condition associated with seizure is tonic-clonic seizures, such as tonic-clonic seizures associated with epilepsy.

### Subject/Patient

The method of treatment typically comprises administering Form A, B, C or E, to a subject or patient.

The subject/patient may be a chordate, a vertebrate, a mammal, a placental mammal, a marsupial (e.g., kangaroo, wombat), a rodent (e.g., a guinea pig, a hamster, a rat, a mouse), murine (e.g., a mouse), a lagomorph (e.g., a rabbit), avian (e.g., a bird), canine (e.g., a dog), feline (e.g., a cat), equine (e.g., a horse), porcine (e.g., a pig), ovine (e.g., a sheep), bovine (e.g., a cow), a primate, simian (e.g., a monkey or ape), a monkey (e.g., marmoset, baboon), an ape (e.g., gorilla, chimpanzee, orang-utan, gibbon), or a human.

The subject/patient may be any of its forms of development, for example, the subject/patient may be an infant or child.

In a preferred embodiment, the subject/patient is a human, more preferably an adult human.

The subject/patient may also be a non-human mammal used in laboratory research, such as a rodent. Rodents include rats, mice, guinea pigs and chinchillas.

### Routes of Administration

The method of treatment may comprise administering Form A, B, C or E to a subject by any convenient route of administration, whether systemically/peripherally or topically (i.e., at the site of desired action).

The route of administration may be oral (e.g., by ingestion); buccal; sublingual; transdermal (including, e.g., by a patch, plaster, etc.); transmucosal (including, e.g., by a patch, plaster, etc.); intranasal (e.g., by nasal spray); ocular (e.g., by eyedrops); pulmonary (e.g., by inhalation or insufflation therapy using, e.g., via an aerosol, e.g., through the mouth or nose); rectal (e.g., by suppository or enema); vaginal (e.g., by pessary); parenteral, for example, by injection or infusion, including subcutaneous, intradermal, intramuscular, intravenous, intraarterial, intracardiac, intrathecal, intraspinal, intracapsular, subcapsular, intraorbital, intraperitoneal, intratracheal, subcuticular, intraarticular, subarachnoid, and intrasternal; or by implant of a depot or reservoir, for example, subcutaneously or intramuscularly.

### Dosages

The method of treatment typically comprises administering a therapeutically effective amount of Form A, B, C or E to a subject.

Appropriate dosages of the crystalline form of compound 1, such as Form A, B, C or E, as well as pharmaceutical compositions comprising the crystalline form of compound 1, such as Form A, B, C or E, can vary from patient to patient. Determining the optimal dosage will generally involve balancing the level of therapeutic benefit against any risk or deleterious side effects. The selected dosage level will depend on a variety of factors including, but not limited to, the activity of the particular crystalline form, the route of administration, the time of administration, the rate of excretion of the compound, the duration of the treatment, other active agents, compounds, and/or materials used in combination, the severity of the condition, and the species, sex, age, weight, condition, general health, and prior medical history of the patient. The dosage and route of administration will ultimately be at the discretion of the clinician, although generally the dosage will be selected to achieve local concentrations at the site of action which achieve the desired effect without causing substantial harmful or deleterious side-effects.

Administration can be effected in one dose, continuously or intermittently (e.g., in divided doses at appropriate intervals) throughout the course of treatment. Single or multiple administrations can be carried out with the dose level and pattern being selected by the treating clinician.

### Other Aspects and Embodiments

Each and every compatible combination of the embodiments described above is explicitly discloses herein, as if each and every combination was individually and explicitly recited.

Carious further aspects and embodiment of the present invention will be apparent to those skilled in the arti in view of the present disclosure.

Where used, “and/or” is to be taken as a specific disclosure of each of the relevant components or features alone as well as a specific disclosure of the combination of the components or features. For example, “A and/or B” is to be taken as specific disclosure of each of i) A, ii) B, and ii) A and B, just as if each were set out individually.

Unless context dictates otherwise, the descriptions and definitions of the features set out above are not limited to any particular aspect or embodiment of the invention and apply equally to all aspects ad embodiments which are described.

### Definitions

The following definitions are provided in order to aid understanding of the invention.

A_{w} is water activity.

Epilepsy is considered to be a disease of the brain defined by any of the following conditions: (1) At least two unprovoked (or reflex) seizures occurring >24 h apart; (2) one unprovoked (or reflex) seizure and a probability of further seizures similar to the general recurrence risk (at least 60%) after two unprovoked seizures, occurring over the next 10 years; (3) diagnosis of an epilepsy syndrome (A practical clinical definition of epilepsy by the International League Against Epilepsy (ILAE), 2014).

The term “focal seizure” (“focal onset seizure”) refers to seizures originating within networks limited to one hemisphere. They may be discretely localized or more widely distributed. Focal seizures may originate in subcortical structures (Operational Classification of Seizure Types by the ILAE, 2017).

The term “generalized seizure” (“generalized onset seizures”) refers to seizures conceptualized as originating at some point within the brain and rapidly engaging bilaterally distributed networks (Operational Classification of Seizure Types by the ILAE, 2017).

The term “pharmaceutically acceptable” pertains to compounds, ingredients, materials, compositions, dosage forms, etc., which are, within the scope of sound medical judgment, suitable for use in contact with the tissues of the subject in question (e.g., human) without excessive toxicity, irritation, allergic response, or other problem or complication, commensurate with a reasonable benefit/risk ratio. Each ingredient (e.g. carrier, diluent, excipient, etc.) must also be “acceptable” in the sense of being compatible with the other ingredients of the composition.

The term “therapeutically-effective amount” pertains to that amount of a compound, or a material, composition or dosage form comprising a compound, which is effective for producing some desired therapeutic effect, commensurate with a reasonable benefit/risk ratio, when administered in accordance with a desired treatment regimen.

A “tonic-clonic seizure” occurs in two phases, a tonic phase typically involving muscle stiffening and loss of consciousness, and a clonic phase typically involving rhythmically jerking of the limbs.

### Worked Examples

Certain aspects and embodiments of the invention will not be illustrated by way of example and with reference to the figures described above.

### Analytical Methods

### X-ray Powder Diffraction (XRPD)

XRPD analyses were performed using a Panalytical Xpert Pro diffractometer equipped with a Cu X-ray tube and a Pixcel detector system. The samples were analysed at ambient temperature in transmission mode and held between low density PVC. The XRPD program parameters were: range 3-40 °2θ, step size 0.013°, counting time 99 sec, ˜22 min run time. Samples were spun at 60 rpm during data collection. XRPD patterns were sorted and manipulated using HighScore Plus 2.2c/v4.9 software.

XRPD analyses were also performed using a Panalytical Empyrean diffractometer equipped with a Cu X-ray tube and a PIXcel 1D-Medipix3 detector system. The samples were analysed at ambient temperature in transmission mode and held between low density PVC. The XRPD program parameters were: range 4-40 °2θ, step size 0.01313°, counting time 23 sec, ˜5 min run time. Samples were spun at 60 rpm during data collection. XRPD patterns were sorted, manipulated and indexed using HighScore Plus 2.2c/v4.9 software.

Differential Scanning Calorimetry (DSC)

DSC analyses were carried out on a Perkin Elmer DSC8500 Differential Scanning Calorimeter. Accurately weighed samples were placed in crimped aluminium pans (i.e. closed but not gas tight). Each sample was heated under nitrogen at a rate of 10° C./minute to a maximum of 250° C. Indium metal was used as the calibration standard. Temperatures were reported at the transition onset to the nearest 0.01 degree.

### Hyper Differential Scanning Calorimetry (Hyper DSC)

Hyper DSC analyses were carried out on a Perkin Elmer DSC8500 Differential Scanning Calorimeter. Accurately weighed samples were placed in crimped aluminium pans (i.e. closed but not gas tight). Each sample was heated and cooled under nitrogen over two cycles at a rate of 300° C./minute using a set temperature range of −50 to 250° C. Indium metal was used as the calibration standard.

### Thermogravimetric Differential Thermal Analysis (TG/DTA)

Thermogravimetric analyses were carried out on a Mettler Toledo TGA/DSC1 STARe. The calibration standards were indium and tin. Samples were placed in an aluminium sample pan, inserted into the TG furnace and accurately weighed. Under a stream of nitrogen at a rate of 10° C./minute, the heat flow signal was stabilised for one minute at 30° C., prior to heating to 300° C.

### Nuclear Magnetic Resonance spectroscopy (NMR)

NMR analysis was carried out on a Bruker 500 MHz instrument in deuterated methanol or d₆-DMSO.

### Optical Microscopy

Microscopy analyses were carried out using an Olympus BX51 stereomicroscope with cross-polarised light and a 1st order red compensator plate. Photomicrographic images were captured using a ColorView Illu digital camera and SynchronizIR basic V5.0 imaging software with objective lens magnification of ×10.

### Example 1: Chemical Synthesis of Compound 1

Scheme 1, below, describes three stages of the process which was used to produce the CBD derivative compound 1, formed via a number of intermediates.

(1S,4R)-1-Methyl-4-(prop-1-en-2-yl)cyclohex-2-en-1-ol (Menthadienol) was coupled with Phloroglucinol using BF₃—OEt₂ to give the trihydroxybenzene derivative in moderate yield.

Treatment with trifluoromethanesulphonic anhydride in a regioselective triflation gave the aryl triflate in a good yield.

The aryl triflate and pyrazole boronate were coupled in a Suzuki reaction, catalysed by palladium. The title compound was afforded in a good yield.

Analytical data of compound 1 is: ¹H NMR (500 MHZ, DMSO) δ8.88 (s, 2H), 7.81 (s, 1H), 7.53 (s, 1H), 6.32 (s, 2H), 5.11 (m, 1H), 4.51 (m, 1H), 4.42 (dd, J=3.0, 1.6 Hz, 1H), 3.86 (m, 1H), 3.80 (s, 3H), 3.05 (ddd, J=13.2, 10.5, 2.8 Hz, 1H), 2.09 (m, 1H), 1.97 (m, 1H), 1.69 (m, 1H), 1.63 (m, 1H), 1.56 (s, 3H), 1.55 (s, 3H). MS (ES+): m/z 325.2 (M+1). HPLC purity 99.2%.

Scheme 1: Synthesis of compound 1

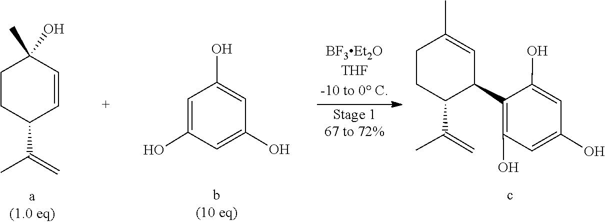 [SMILES: C=C(C)[C@H]1C=C[C@@](C)(O)CC1 ; Oc1cc(O)cc(O)c1 ; C=C(C)[C@@H]1CCC(C)=C[C@H]1c1c(O)cc(O)cc1O]

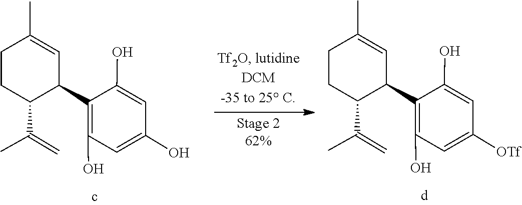 [SMILES: C=C(C)[C@@H]1CCC(C)=C[C@H]1c1c(O)cc(O)cc1O ; C=C(C)[C@@H]1CCC(C)=C[C@H]1c1c(O)cc(OS(=O)(=O)C(F)(F)F)cc1O]

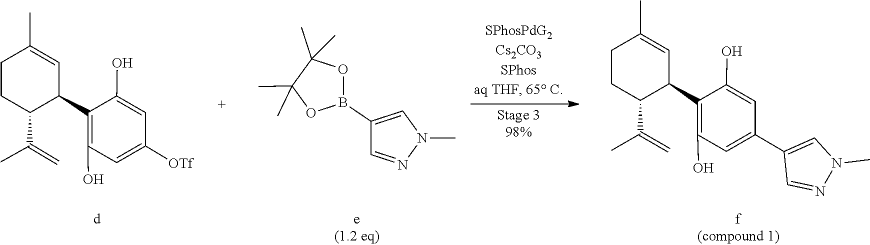

| Compound | Name |
|---|---|
| 1 | Menthadienol |
| b | Benzene-1,3,5-triol (Phloroglucinol) |
| c | (1′R,2′R)-5′-Methyl-2′-(prop-1-en-2-yl)-1′,2′,3′,4′-tetrahydro-[1,1′-biphenyl]-2,4,6-triol |
| d | (1′R,2′R)-2,6-Dihydroxy-5′-methyl-2′-(prop-1-en-2-yl)-1′,2′,3′,4′-tetrahydro-[1,1′-biphenyl]-4-yl trifluoromethanesulfonate |
| e | 1-Methyl-4-(4,4,5,5-tetramethyl-1,3,2-dioxaborolan-2-yl)-1H-pyrazole |
| f | (1′R,2′R)-5′-methyl-4-(1-methyl-1H-pyrazol-4-yl)-2′-(prop-1-en-2-yl)-1′,2′,3′,4′-tetrahydro-[1,1′-biphenyl]-2,6-diol |

### Example 2: Preparation of Amorphous Compound 1

Compound 1 was subjected to a melt quench procedure order to ensure a fully amorphous material for comparison. Compound 1 (20.1 mg) was added to a HPLC vial and flushed with N₂. This was heated to ˜190° C. for 2 minutes and was quickly immersed in an ice/water bath to form a glassy solid material.

### Characterisation of Amorphous Compound 1

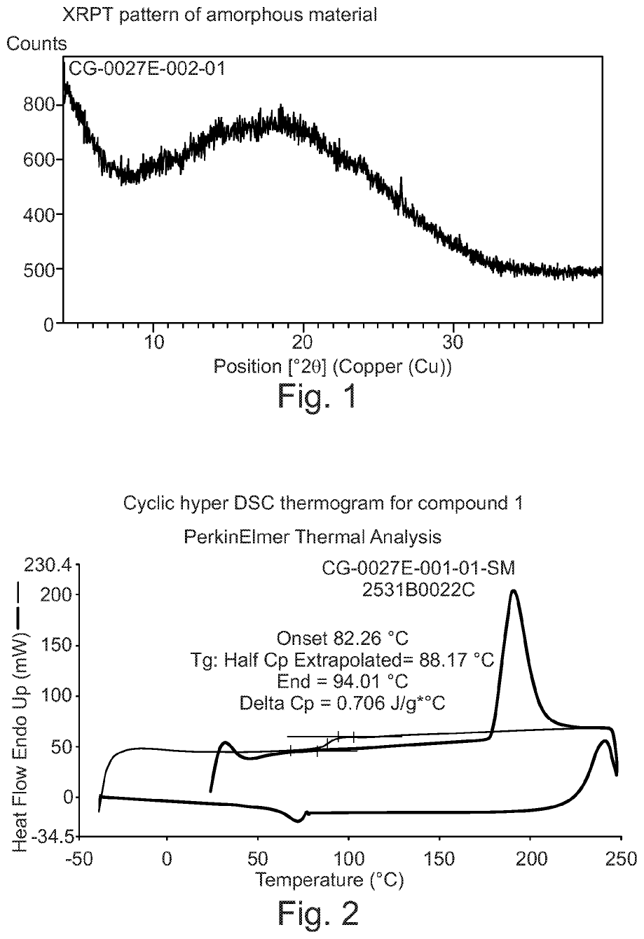

The XRPD pattern obtained for amorphous compound 1 is shown in FIG. 1. XRPD analysis displayed a halo pattern indicative of X-ray amorphous material.

The ¹H NMR spectrum of the material conformed to the molecular structure and degradation was not observed.

A cyclic hyper DSC experiment was performed in order to generate amorphous material from fast cooling of molten compound 1 and determine the temperature of glass transition (T_{g}) during the re-heat cycle. A cyclic thermogram, shown in FIG. 2, demonstrates the presence of a T_{g}, detected in the reheat cycle, at 88° C. (half-height cₚ value).

### Example 3: Preparation of Form A by Sonication

Amorphous compound 1 (˜20 mg) was added to a vial with 20 μL of heptane to form a paste. The mixture was sonicated at 70% intensity using a Cole-Parmer 130 W ultrasonic processor using a pulsed program. The wet paste was recovered.

### Characterisation of Form A

The XRPD pattern obtained for Form A compound 1 is shown in FIG. 3. The XRPD pattern is indicative of a crystalline material. The XRPD peak listing data is given in Table 1.

TABLE 1
Observed XRPD data for Compound 1 Form A

| | Pos. | FWHM | Backgr. | d-spacing | Height | Rel. |
|---|---|---|---|---|---|---|
| No. | [°2θ] | Left [°2θ] | [cts] | [Å] | [cts] | Int. [%] |
| 1 | 4.82 | 0.0384 | 77.39 | 18.33 | 294.60 | 7.2 |
| 2 | 9.63 | 0.0384 | 30.26 | 9.18 | 143.88 | 3.5 |
| 3 | 12.21 | 0.0512 | 32.87 | 7.25 | 807.34 | 19.6 |
| 4 | 12.91 | 0.0640 | 33.68 | 6.86 | 4120.07 | 100.0 |
| 5 | 13.90 | 0.0512 | 31.87 | 6.37 | 264.88 | 6.4 |
| 6 | 14.46 | 0.0512 | 32.53 | 6.13 | 88.50 | 2.2 |
| 7 | 14.92 | 0.0640 | 31.15 | 5.94 | 1410.89 | 34.2 |
| 8 | 16.22 | 0.0512 | 29.84 | 5.46 | 114.92 | 2.8 |
| 9 | 17.71 | 0.0640 | 32.13 | 5.01 | 544.72 | 13.2 |
| 10 | 17.89 | 0.0768 | 32.30 | 4.96 | 1838.66 | 44.6 |
| 11 | 19.15 | 0.0768 | 36.88 | 4.63 | 1039.92 | 25.2 |
| 12 | 19.50 | 0.0384 | 36.23 | 4.55 | 268.48 | 6.5 |
| 13 | 20.20 | 0.0768 | 31.19 | 4.40 | 672.80 | 16.3 |
| 14 | 20.72 | 0.0640 | 27.31 | 4.29 | 110.38 | 2.7 |
| 15 | 22.94 | 0.0640 | 31.45 | 3.88 | 133.34 | 3.2 |
| 16 | 23.34 | 0.0512 | 34.24 | 3.81 | 95.83 | 2.3 |
| 17 | 24.03 | 0.0649 | 32.00 | 3.70 | 1329.64 | 32.3 |
| 18 | 24.16 | 0.0568 | 33.90 | 3.68 | 464.26 | 11.3 |
| 19 | 24.53 | 0.0512 | 40.06 | 3.63 | 417.11 | 10.1 |
| 20 | 25.12 | 0.0512 | 29.72 | 3.54 | 430.03 | 10.4 |
| 21 | 25.96 | 0.0768 | 27.84 | 3.43 | 43.32 | 1.1 |
| 22 | 26.34 | 0.0768 | 27.45 | 3.38 | 43.32 | 1.1 |
| 23 | 26.96 | 0.0512 | 30.44 | 3.31 | 287.00 | 7.0 |
| 24 | 27.54 | 0.0640 | 33.49 | 3.24 | 628.62 | 15.3 |
| 25 | 27.87 | 0.0384 | 32.58 | 3.20 | 259.00 | 6.3 |
| 26 | 28.17 | 0.0512 | 29.21 | 3.17 | 59.43 | 1.4 |
| 27 | 28.40 | 0.0384 | 25.83 | 3.14 | 96.46 | 2.3 |
| 28 | 29.13 | 0.0640 | 26.08 | 3.07 | 119.08 | 2.9 |
| 29 | 29.51 | 0.0768 | 28.66 | 3.03 | 18.61 | 0.5 |
| 30 | 30.11 | 0.1535 | 23.02 | 2.97 | 36.59 | 0.9 |
| 31 | 30.54 | 0.0768 | 16.99 | 2.93 | 46.14 | 1.1 |
| 32 | 31.14 | 0.1023 | 16.61 | 2.87 | 58.29 | 1.4 |
| 33 | 32.39 | 0.1023 | 15.84 | 2.76 | 23.89 | 0.6 |
| 34 | 34.31 | 0.0768 | 15.30 | 2.61 | 36.88 | 0.9 |
| 35 | 35.26 | 0.1023 | 14.87 | 2.55 | 32.36 | 0.8 |
| 36 | 35.95 | 0.0768 | 17.51 | 2.50 | 56.32 | 1.4 |
| 37 | 36.74 | 0.0512 | 18.10 | 2.45 | 121.83 | 3.0 |
| 38 | 37.75 | 0.1535 | 12.15 | 2.38 | 14.47 | 0.4 |
| 39 | 39.55 | 0.1535 | 21.06 | 2.28 | 28.72 | 0.7 |

Form A material was analysed by ¹H NMR spectroscopy in d₆-DMSO. The ¹H NMR spectrum of Form A material conformed to the molecular structure and solvent was not detected.

Thermogravimetric/Differential Thermal Analysis (TG/DTA) was performed to determine the thermal profile and associated percentage weight changes of compound 1 (FIG. 4).

A weight loss of 0.27% was noted from ˜30° C. to 211° C., suggesting minimal moisture or solvent content, indicating Form A to be an anhydrous material. A second weight loss at temperatures greater than 220° C. corresponds to the initiation of decomposition of the material. An endotherm assigned to the melt of Form A was observed at onset temperature 179° C. followed by a second endotherm at onset temperature 194° C.

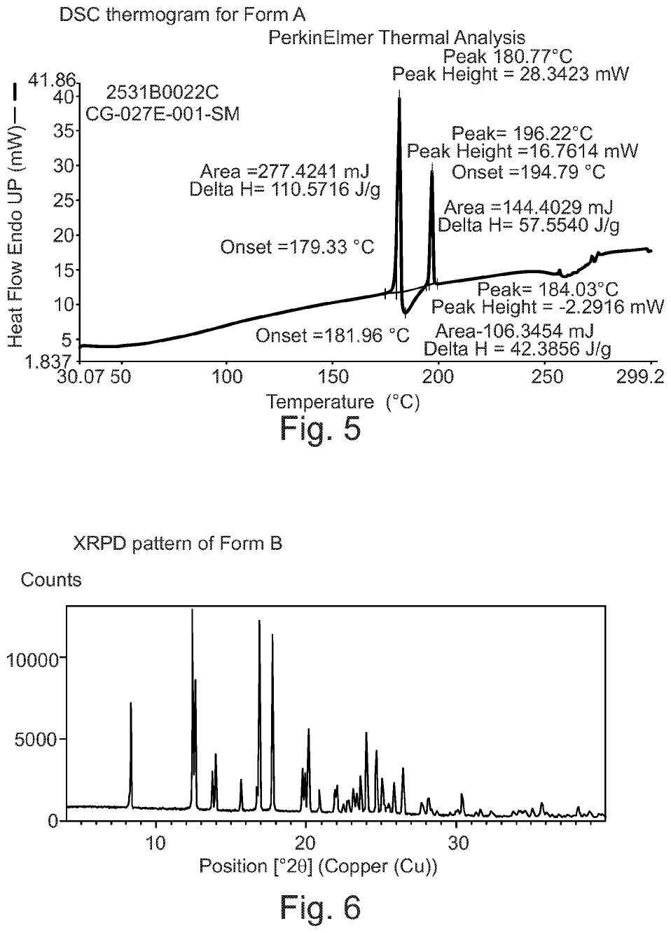

The DSC thermogram obtained for Form A material at 10° C./min is shown in FIG. 5 and is in agreement with the TG/DTA data, showing onset of melt at 179° C. quickly followed by recrystallization and second melt with onset at 194° C.

Based on this analysis, it is likely that crystalline Form A material is anhydrous.

### Example 4: Preparation of Form B by Slurry Process

Compound 1 (amorphous) was added to ethyl acetate until undissolved solids remained at the desired temperature (20° C.). The vial was sealed, and the slurry was maintained at the desired temperature and agitated by magnetic stirring for 7 days. The solid was isolated by drying on filter paper under a flow of air for 5-10 minutes prior to analysis.

Compound 1 (amorphous) was added to a cyclohexane until undissolved solids remained at the desired temperature (50° C.). The vial was sealed, and the slurry was maintained at the desired temperature and agitated by magnetic stirring for 3 days at 50° C. The solid was isolated by drying on filter paper under a flow of air for 5-10 minutes prior to analysis.

Both slurry methods were found to yield Form B.

### Characterisation of Form B

The XRPD pattern obtained for Form B compound 1 is shown in FIG. 6. The XRPD pattern is indicative of a crystalline material. The XRPD peak listing data is given in Table 2.

TABLE 2
Observed XRPD data for Compound 1 Form B

| | Pos. | FWHM | Backgr. | d-spacing | Height | Rel. |
|---|---|---|---|---|---|---|
| No. | [°2θ] | Left [°2θ] | [cts] | [Å] | [cts] | Int. [%] |
| 1 | 8.33 | 0.0640 | 810.45 | 10.61 | 6647.67 | 53.2 |
| 2 | 12.45 | 0.0640 | 751.56 | 7.11 | 12498.80 | 100.0 |
| 3 | 12.61 | 0.0640 | 746.81 | 7.02 | 8087.11 | 64.7 |
| 4 | 13.74 | 0.0640 | 675.29 | 6.44 | 2369.42 | 19.0 |
| 5 | 13.96 | 0.0768 | 664.88 | 6.34 | 3488.36 | 27.9 |
| 6 | 15.65 | 0.0768 | 629.52 | 5.66 | 1939.12 | 15.5 |
| 7 | 16.70 | 0.0640 | 663.34 | 5.31 | 1380.62 | 11.1 |
| 8 | 16.88 | 0.0768 | 668.76 | 5.25 | 11866.67 | 94.9 |
| 9 | 17.75 | 0.0895 | 651.53 | 5.00 | 11074.36 | 88.6 |
| 10 | 19.73 | 0.0895 | 592.56 | 4.50 | 2634.60 | 21.1 |
| 11 | 19.91 | 0.0768 | 592.45 | 4.46 | 2378.73 | 19.0 |
| 12 | 20.14 | 0.0895 | 589.65 | 4.41 | 5144.98 | 41.2 |
| 13 | 20.88 | 0.0640 | 549.82 | 4.25 | 1392.77 | 11.1 |
| 14 | 21.91 | 0.0640 | 534.68 | 4.06 | 1324.07 | 10.6 |
| 15 | 22.06 | 0.0895 | 532.82 | 4.03 | 1699.18 | 13.6 |
| 16 | 22.47 | 0.1023 | 535.14 | 3.96 | 454.51 | 3.6 |
| 17 | 22.74 | 0.0624 | 551.40 | 3.91 | 645.42 | 5.2 |
| 18 | 22.85 | 0.0768 | 554.66 | 3.89 | 655.38 | 5.2 |
| 19 | 23.16 | 0.1023 | 569.24 | 3.84 | 1388.32 | 11.1 |
| 20 | 23.36 | 0.1151 | 577.27 | 3.81 | 1098.08 | 8.8 |
| 21 | 23.62 | 0.1023 | 585.42 | 3.77 | 2183.04 | 17.5 |
| 22 | 23.99 | 0.1151 | 576.67 | 3.71 | 4600.89 | 36.8 |
| 23 | 24.66 | 0.1023 | 547.71 | 3.61 | 3648.09 | 29.2 |
| 24 | 25.06 | 0.1535 | 548.71 | 3.55 | 2119.82 | 17.0 |
| 25 | 25.51 | 0.0640 | 527.36 | 3.49 | 505.92 | 4.1 |
| 26 | 25.85 | 0.1151 | 490.18 | 3.45 | 1885.84 | 15.1 |
| 27 | 26.44 | 0.1279 | 443.20 | 3.37 | 2694.57 | 21.6 |
| 28 | 27.67 | 0.0640 | 406.75 | 3.22 | 649.87 | 5.2 |
| 29 | 28.10 | 0.0624 | 406.20 | 3.17 | 900.95 | 7.2 |
| 30 | 28.18 | 0.0640 | 405.20 | 3.17 | 946.86 | 7.6 |
| 31 | 28.37 | 0.1023 | 394.94 | 3.15 | 275.37 | 2.2 |
| 32 | 28.71 | 0.1151 | 360.54 | 3.11 | 215.47 | 1.7 |
| 33 | 29.55 | 0.0895 | 334.05 | 3.02 | 142.14 | 1.1 |
| 34 | 29.94 | 0.0768 | 342.02 | 2.98 | 241.03 | 1.9 |
| 35 | 30.10 | 0.1023 | 339.39 | 2.97 | 345.03 | 2.8 |
| 36 | 30.33 | 0.0512 | 332.92 | 2.95 | 1166.71 | 9.3 |
| 37 | 31.32 | 0.1023 | 296.16 | 2.86 | 204.46 | 1.6 |
| 38 | 31.58 | 0.0768 | 293.84 | 2.83 | 347.62 | 2.8 |
| 39 | 32.29 | 0.1279 | 284.17 | 2.77 | 290.76 | 2.3 |
| 40 | 33.76 | 0.0768 | 303.34 | 2.65 | 288.14 | 2.3 |
| 41 | 34.13 | 0.0640 | 318.97 | 2.63 | 281.93 | 2.3 |
| 42 | 34.63 | 0.1279 | 312.90 | 2.59 | 274.34 | 2.2 |
| 43 | 35.09 | 0.1279 | 299.79 | 2.56 | 467.13 | 3.7 |
| 44 | 35.66 | 0.0768 | 296.63 | 2.52 | 732.93 | 5.9 |
| 45 | 36.04 | 0.1023 | 286.62 | 2.49 | 167.68 | 1.3 |
| 46 | 36.40 | 0.1279 | 262.94 | 2.47 | 111.91 | 0.9 |
| 47 | 36.80 | 0.1023 | 241.54 | 2.44 | 108.23 | 0.9 |
| 48 | 37.44 | 0.1023 | 245.67 | 2.40 | 93.91 | 0.8 |
| 49 | 38.07 | 0.1151 | 270.02 | 2.36 | 440.33 | 3.5 |
| 50 | 38.49 | 0.1535 | 278.23 | 2.34 | 86.77 | 0.7 |
| 51 | 38.83 | 0.0640 | 269.70 | 2.32 | 278.70 | 2.2 |
| 52 | 39.50 | 0.1535 | 250.99 | 2.28 | 148.05 | 1.2 |

Form B material was analysed by ¹H NMR spectroscopy in d₄-MeOD. The ¹H NMR spectrum of Form B material conformed to the molecular structure and solvent was not detected.

The thermogravimetric/Differential Thermal Analysis (TG/DTA) data for Form B exhibited minimal weight loss below the onset of melt at 195° C. and onset of decomposition above 260° C. (FIG. 7).

Based on this analysis, it is likely that crystalline Form B material is anhydrous.

### Stability of Form B to Milling

A ball mill was used to mimic the effects of grinding that would be experienced during formulation steps such as dry blending and wet granulation. Form B (50 mg) was added to a milling chamber with an agate milling ball. Using a Retsch MM200 mixer mill, the material was milled for 6-10 minutes at a frequency of 25 Hz. Periodically, the milling was stopped and powder that adhered to the milling chamber was scraped down. The resultant milled material was analysed using XRPD.

TABLE 3
Results of milling experiment

| Input Form | Solvent | Conditions | Result | Output Form |
|---|---|---|---|---|
| B | None | mill at 25 Hz, | solid | B (disordered) |
| | | 3 × 2 mins | | |

Form B did not undergo a change in form, but some disorder was detected (FIG. 8).

### Stability of Form B to Compression

A die press was used to mimic the uniaxial stress experienced during formulation steps such as tableting. Form B (50 mg) was added to a KBr pellet die and compressed overnight at ˜370 MPa using a hydraulic press. The resultant solid disc was removed from the press and immediately analysed by XRPD.

TABLE 4
Results of compression experiment

| Input Form | Conditions | Result | Output Form |
|---|---|---|---|
| B | Compression @ | solid | B (slight disorder) |
| | 370 MPa for 24 hrs | | |

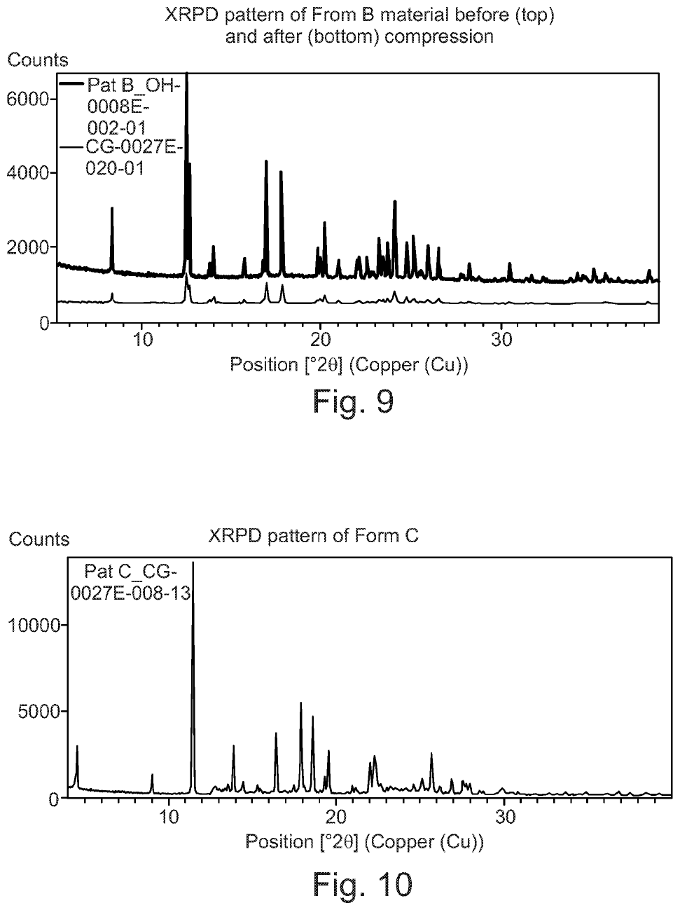

Form B was stable to compression, although there was some introduction of minor disorder by XRPD analysis (FIG. 9).

### Stability of Form B to Humidity Stress

Form B material was exposed to high humidity conditions to determine stability on storage. Approximately 20 mg of Form B material was added to a vial and placed unsealed into a sealed cabinet with a relative humidity of 75% (controlled by a super-saturated salt solution) and a temperature of 40° C. for 8 days. The resultant material was analysed by XRPD.

TABLE 5
Results of humidity stress experiment

| Input Form | Conditions | Result | Output Form |
|---|---|---|---|
| B | 40° C./75% RH for 8 days | solid | B |

Form B was stable to storage under these conditions.

### Example 5: Preparation of Form C by Sonication and Slow Evaporation

Amorphous compound 1 (˜20 mg) was added to a vial with 20 μL of EtOH/water (96:4% v/v; a_{w} ˜0.25) to form a paste. The mixture was sonicated at 70% intensity using a Cole-Parmer 130W ultrasonic processor using a pulsed program. The wet paste was recovered and analysed.

A solution of compound 1 Form A in ethanol was evaporated in a fume hood at ambient temperature (˜20° C.) in a vial. The resulting solid was isolated and analysed.

Both the sonication process and the slow evaporation process provided Form C material.

### Characterisation of Form C

The XRPD pattern obtained for Form C compound 1 is shown in FIG. 10. The XRPD pattern is indicative of a crystalline material. The XRPD peak listing data is given in Table 6.

TABLE 6
Observed XRPD data for Compound 1 Form C

| | Pos. | FWHM | Backgr. | d-spacing | Height | Rel. |
|---|---|---|---|---|---|---|
| No. | [°2θ] | Left [°2θ] | [cts] | [Å] | [cts] | Int. [%] |
| 1 | 4.51 | 0.0384 | 547.66 | 19.59 | 2583.85 | 18.8 |
| 2 | 7.11 | 0.4093 | 350.73 | 12.44 | 25.18 | 0.2 |
| 3 | 9.01 | 0.0512 | 267.49 | 9.82 | 1110.79 | 8.1 |
| 4 | 10.76 | 0.1535 | 238.10 | 8.22 | 76.85 | 0.6 |
| 5 | 11.45 | 0.0640 | 241.80 | 7.73 | 13777.51 | 100.0 |
| 6 | 12.78 | 0.1791 | 304.47 | 6.93 | 325.50 | 2.4 |
| 7 | 13.33 | 0.0768 | 366.97 | 6.64 | 171.84 | 1.3 |
| 8 | 13.52 | 0.0512 | 379.83 | 6.55 | 416.42 | 3.0 |
| 9 | 13.86 | 0.0640 | 382.18 | 6.39 | 2711.88 | 19.7 |
| 10 | 14.40 | 0.0640 | 341.16 | 6.15 | 561.18 | 4.1 |
| 11 | 15.26 | 0.0640 | 323.07 | 5.81 | 394.59 | 2.9 |
| 12 | 15.45 | 0.0768 | 319.53 | 5.74 | 183.54 | 1.3 |
| 13 | 16.39 | 0.0768 | 345.11 | 5.41 | 3466.60 | 25.2 |
| 14 | 17.43 | 0.0768 | 375.67 | 5.09 | 346.56 | 2.5 |
| 15 | 17.86 | 0.0895 | 383.38 | 4.97 | 5205.92 | 37.8 |
| 16 | 18.57 | 0.0895 | 350.48 | 4.78 | 4424.10 | 32.1 |
| 17 | 19.00 | 0.0768 | 329.44 | 4.67 | 101.97 | 0.7 |
| 18 | 19.27 | 0.0768 | 316.38 | 4.61 | 898.23 | 6.5 |
| 19 | 19.51 | 0.1023 | 301.42 | 4.55 | 2456.26 | 17.8 |
| 20 | 20.94 | 0.0895 | 282.12 | 4.24 | 441.69 | 3.2 |
| 21 | 21.13 | 0.0768 | 281.78 | 4.20 | 310.77 | 2.3 |
| 22 | 21.63 | 0.0768 | 304.65 | 4.11 | 39.65 | 0.3 |
| 23 | 21.98 | 0.1151 | 365.29 | 4.04 | 1592.79 | 11.6 |
| 24 | 22.25 | 0.1279 | 396.18 | 4.00 | 2036.47 | 14.8 |
| 25 | 22.63 | 0.1023 | 429.83 | 3.93 | 393.42 | 2.9 |
| 26 | 22.99 | 0.0768 | 442.25 | 3.87 | 153.18 | 1.1 |
| 27 | 23.20 | 0.1535 | 449.26 | 3.83 | 237.18 | 1.7 |
| 28 | 24.11 | 0.1535 | 428.87 | 3.69 | 122.91 | 0.9 |
| 29 | 24.58 | 0.0768 | 407.52 | 3.62 | 377.87 | 2.7 |
| 30 | 25.08 | 0.1151 | 412.64 | 3.55 | 737.75 | 5.4 |
| 31 | 25.66 | 0.1279 | 396.11 | 3.47 | 2246.89 | 16.3 |
| 32 | 26.15 | 0.0512 | 351.81 | 3.41 | 326.02 | 2.4 |
| 33 | 26.83 | 0.1151 | 270.30 | 3.32 | 818.76 | 5.9 |
| 34 | 27.51 | 0.0895 | 235.15 | 3.24 | 706.38 | 5.1 |
| 35 | 27.70 | 0.0768 | 233.88 | 3.22 | 618.93 | 4.5 |
| 36 | 27.91 | 0.0768 | 232.51 | 3.20 | 599.37 | 4.4 |
| 37 | 28.53 | 0.1023 | 215.83 | 3.13 | 186.88 | 1.4 |
| 38 | 28.75 | 0.1279 | 215.94 | 3.11 | 135.50 | 1.0 |
| 39 | 29.87 | 0.1535 | 248.72 | 2.99 | 296.58 | 2.2 |
| 40 | 30.41 | 0.2047 | 248.68 | 2.94 | 59.97 | 0.4 |
| 41 | 30.78 | 0.1023 | 221.50 | 2.90 | 113.38 | 0.8 |
| 42 | 31.89 | 0.3070 | 172.04 | 2.81 | 54.61 | 0.4 |
| 43 | 32.67 | 0.1535 | 170.10 | 2.74 | 82.62 | 0.6 |
| 44 | 33.66 | 0.1279 | 175.68 | 2.66 | 141.43 | 1.0 |
| 45 | 34.41 | 0.8187 | 175.71 | 2.61 | 9.37 | 0.1 |
| 46 | 34.87 | 0.1535 | 177.20 | 2.57 | 170.42 | 1.2 |
| 47 | 36.07 | 0.1279 | 170.26 | 2.49 | 55.82 | 0.4 |
| 48 | 36.80 | 0.2047 | 155.01 | 2.44 | 204.60 | 1.5 |
| 49 | 37.45 | 0.1791 | 134.72 | 2.40 | 155.65 | 1.1 |
| 50 | 38.47 | 0.2558 | 153.77 | 2.34 | 135.54 | 1.0 |
| 51 | 39.22 | 0.2047 | 162.53 | 2.30 | 68.39 | 0.5 |

Form C material was analysed by ¹H NMR spectroscopy in d₄-MeOD. The ¹H NMR spectrum of Form C material conformed to the molecular structure and contained ethanol (1.0 mol eq.).

The thermogravimetric/Differential Thermal Analysis (TG/DTA) data for Form C displayed an 11% weight loss from 42-114° C., which equates to 1 mol eq. of EtOH (FIG. 11). Onset of melt was noted at 178° C., quickly followed by recrystallisation and a second melt with onset at 194° C. This suggests Form C material desolvates to Form A material, followed by melt of Form A, recrystallisation and melt of Form B material.

Form C material converted to a Form A when retested after 6 days storage at ambient conditions.

Based on this analysis, it is likely that Form C material is an ethanol monosolvate.

### Example 6: Preparation of Form E by Slurry Process and Slow Evaporation

Compound 1 (amorphous) was added to IMS (5% MeOH in EtOH) until undissolved solids remained at the desired temperature (20° C.). The vial was sealed and the slurry was maintained at the desired temperature and agitated by magnetic stirring for 7 days. The solid was isolated by drying on filter paper under a flow of air for 5-10 minutes prior to analysis.

A solution of compound 1 Form A prepared in IMS (5% MeOH in EtOH) was evaporated in a fume hood at ambient temperature (˜20° C.) in a vial. The resulting was isolated and analysed.

Both the slurry process and the slow evaporation process provided Form E material.

### Characterisation of Form E

The XRPD pattern obtained for Form E compound 1 is shown in FIG. 12. The XRPD pattern is indicative of a crystalline material. The XRPD peak listing data is given in Table 7.

TABLE 7
Observed XRPD data for Compound 1 Form E

| | Pos. | FWHM | Backgr. | d-spacing | Height | Rel. |
|---|---|---|---|---|---|---|
| No. | [°2θ] | Left [°2θ] | [cts] | [Å] | [cts] | Int. [%] |
| 1 | 4.51 | 0.0512 | 564.34 | 19.58 | 2045.73 | 28.2 |
| 2 | 9.02 | 0.0384 | 254.68 | 9.81 | 969.12 | 13.4 |
| 3 | 11.48 | 0.0640 | 211.01 | 7.71 | 7253.88 | 100.0 |
| 4 | 12.91 | 0.0640 | 237.45 | 6.86 | 387.13 | 5.3 |
| 5 | 13.53 | 0.0512 | 259.07 | 6.54 | 333.74 | 4.6 |
| 6 | 13.89 | 0.0640 | 252.97 | 6.38 | 1397.33 | 19.3 |
| 7 | 14.42 | 0.0768 | 230.25 | 6.14 | 220.85 | 3.0 |
| 8 | 14.93 | 0.0768 | 228.43 | 5.94 | 119.40 | 1.7 |
| 9 | 15.29 | 0.1023 | 222.23 | 5.79 | 185.15 | 2.6 |
| 10 | 15.46 | 0.0768 | 217.99 | 5.73 | 113.18 | 1.6 |
| 11 | 16.41 | 0.0640 | 225.35 | 5.40 | 1564.44 | 21.6 |
| 12 | 17.46 | 0.0768 | 257.67 | 5.08 | 130.85 | 1.8 |
| 13 | 17.89 | 0.0895 | 263.31 | 4.96 | 2248.43 | 31.0 |
| 14 | 18.07 | 0.0512 | 262.04 | 4.91 | 239.24 | 3.3 |
| 15 | 18.60 | 0.0768 | 254.36 | 4.77 | 1917.67 | 26.4 |
| 16 | 19.03 | 0.0768 | 245.28 | 4.66 | 94.87 | 1.3 |
| 17 | 19.29 | 0.0640 | 236.55 | 4.60 | 537.71 | 7.4 |
| 18 | 19.54 | 0.0895 | 222.75 | 4.54 | 1115.19 | 15.4 |
| 19 | 20.96 | 0.0768 | 195.53 | 4.24 | 266.14 | 3.7 |
| 20 | 21.17 | 0.0768 | 195.50 | 4.20 | 162.89 | 2.3 |
| 21 | 22.02 | 0.0768 | 230.70 | 4.04 | 911.67 | 12.6 |
| 22 | 22.29 | 0.1023 | 245.88 | 3.99 | 901.34 | 12.4 |
| 23 | 22.64 | 0.0640 | 261.85 | 3.93 | 343.55 | 4.7 |
| 24 | 23.05 | 0.0768 | 264.96 | 3.86 | 117.32 | 1.6 |
| 25 | 24.12 | 0.3070 | 238.91 | 3.69 | 69.75 | 1.0 |
| 26 | 24.59 | 0.0640 | 241.19 | 3.62 | 292.50 | 4.0 |
| 27 | 25.11 | 0.0640 | 243.72 | 3.55 | 512.11 | 7.1 |
| 28 | 25.69 | 0.0768 | 224.90 | 3.47 | 944.83 | 13.0 |
| 29 | 26.19 | 0.1023 | 192.03 | 3.40 | 146.19 | 2.0 |
| 30 | 26.89 | 0.0895 | 170.05 | 3.32 | 390.29 | 5.4 |
| 31 | 27.56 | 0.0640 | 154.01 | 3.24 | 300.15 | 4.1 |
| 32 | 27.76 | 0.0768 | 152.08 | 3.21 | 309.23 | 4.3 |
| 33 | 27.95 | 0.0640 | 149.82 | 3.19 | 268.89 | 3.7 |
| 34 | 28.56 | 0.1023 | 138.03 | 3.13 | 142.19 | 2.0 |
| 35 | 29.89 | 0.1791 | 153.16 | 2.99 | 90.79 | 1.3 |
| 36 | 31.89 | 0.2047 | 105.80 | 2.81 | 36.74 | 0.5 |
| 37 | 33.73 | 0.1535 | 108.64 | 2.66 | 46.84 | 0.7 |
| 38 | 34.95 | 0.1535 | 101.44 | 2.57 | 64.41 | 0.9 |
| 39 | 36.16 | 0.1535 | 109.32 | 2.48 | 25.41 | 0.4 |
| 40 | 36.83 | 0.1279 | 104.75 | 2.44 | 78.74 | 1.1 |
| 41 | 37.52 | 0.1023 | 91.20 | 2.40 | 75.53 | 1.0 |
| 42 | 38.58 | 0.3070 | 95.25 | 2.33 | 54.06 | 0.8 |
| 43 | 39.28 | 0.1535 | 95.49 | 2.29 | 67.38 | 0.9 |

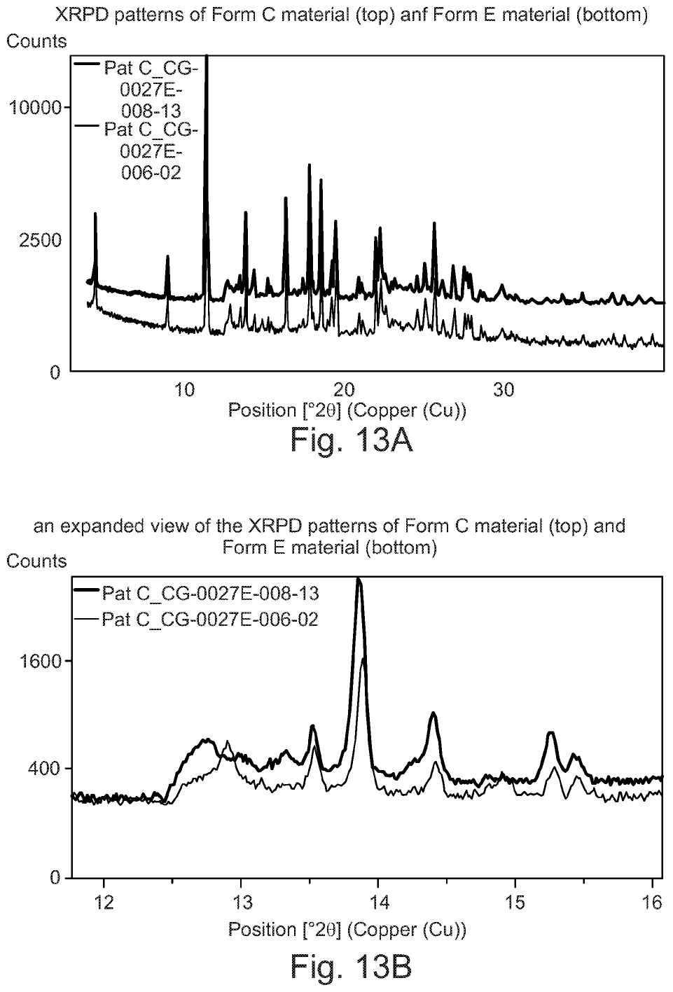

The XRPD data indicated that Form E material was very similar to Form C material (FIGS. 13A and 13B).

Based on this analysis, it is likely that Form E material is a solvate.

### Example 7: Evaluation of Compound 1 for Anticonvulsant Activity using the Maximal Electroshock Seizure Threshold (MEST) Test in the Mouse

The efficacy of compound 1 was tested in a mouse model of generalised seizure, the maximal electroshock seizure threshold (MEST) test.

The maximal electroshock seizure threshold (MEST) test is widely utilized preclinically to evaluate pro-or anti-convulsant properties of test compounds (Loscher et al., 1991).

In the MEST test the ability of a drug to alter the seizure threshold current required to induce hind limb tonic extensor convulsions is measured according to an “up and down” method of shock titration (Kimball et al., 1957). An increase in seizure threshold is indicative of anti-convulsant effect. Antiepileptic drugs including the sodium channel blockers (e.g. lamotrigine) with clinically proven efficacy against generalised tonic-clonic seizures all exhibit anti-convulsant properties in this test in the mouse.

Conversely, a reduction in seizure threshold is indicative of a pro-convulsant effect as observed with known convulsant agents such as picrotoxin.

The ability of a test compound to alter the stimulus intensity, expressed as current (mA), required to induce the presence of tonic hind limb extensor convulsions, is assessed in the MEST. The outcome of the presence (+) or absence (0) of tonic hind limb extensor convulsions observed from a current to produce tonic hind limb extension in 50% of animals in the treatment group (CC50) determines the seizure threshold for the treatment group and the effects were then compared to the CC50 of the vehicle control group.

### Methods

### Study Details

Naïve mice were acclimatised to the procedure room in their home cages for up to 7 days, with food and water available ad libitum.

All animals were weighed at the beginning of the study and randomly assigned to treatment groups based on a mean distribution of body weight across groups. All animals were dosed at 10 mL/kg via intraperitoneal (i.p) injection, with either vehicle, test compound at 2, 20 or 200 mg/kg or diazepam at 2.5 mg/kg.

Animals were individually assessed for the production of a tonic hind limb extensor convulsion at 30 min post-dose for vehicle, 30 min post-dose for test compound and 30 min post-dose for diazepam, from a single electroshock.

The first animal within a treatment group was given a shock at the expected or estimated CC₅₀ current. For subsequent animals, the current was lowered or raised depending on the convulsions outcome from the preceding animal, in intervals of 5 mA.

Data generated from each treatment group were used to calculate the CC₅₀+SEM values for the treatment group.

### Test Compounds

Vehicle: (5% ethanol, 10% solutol, 85% Saline) was prepared as follows: 1 mL of ethanol, 2 mL of solutol were warmed to 60° C., in 17 mL of saline (1:2:17).

Positive control: diazepam was used at 2.5 mg/kg.

The test compound (compound 1) was administered at 2, 20 and 200 mg/kg (i.p.) in a 1:2:17 ethanol: solutol: 0.9% saline formulation.

### Sample Collection

Each animal was humanely killed immediately after production of a convulsion by destruction of the brain from striking the cranium, followed by the confirmation of permanent cessation of the circulation from decapitation under The Humane Killing of Animals under Schedule 1 to the Animals (Scientific Procedures) Act 1986. Terminal blood and brain collection were performed following decapitation.

Blood was collected in Lithium-heparin tubes and centrifuged at 4° C. for 10 minutes at 1500×g. The resulting plasma was removed (>100 μL) and split into 2 aliquots of 0.5 mL Eppendorf tubes containing 100 μL of ascorbic acid (100 mg/mL) for stabilisation. Brains were removed, washed in saline and halved. Each half was placed into separate 2 mL screw cap cryovials, weighed and frozen on cardice.

### Statistical Analysis

The data for each treatment group were recorded as the number of +'s and 0's at each current level employed and this information is then used to calculate the CC₅₀ value (current required for 50% of the animals to show seizure behaviour)±standard error.

Test compound effects were also calculated as percentage change in CC₅₀ from the vehicle control group.

Significant difference between drug-treated animals and controls were assessed according to Litchfield and Wilcoxon (1949).

### Results

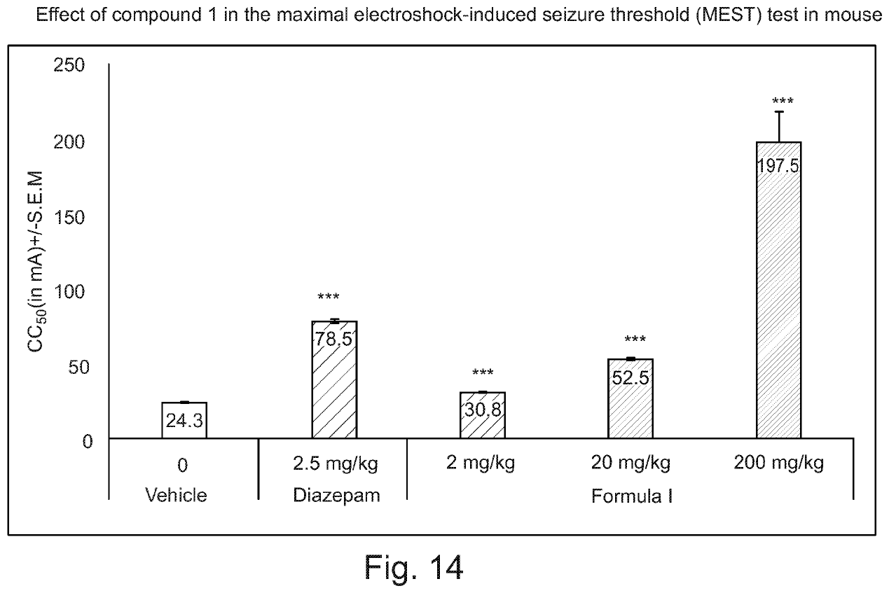

FIG. 14 and Table 8 describe the data produced in this experiment.

In the vehicle group, the CC₅₀ value was calculated to be 24.3 mA.

In the diazepam (2.5 mg/kg) treated group, administered i.p. 30 minutes before the test, the CC₅₀ value was 78.5 mA. This result was statistically significant (p<0.001) compared to the vehicle control. One animal in the diazepam group, was not dosed due to welfare issues from fighting.

In the test compound treatment group, administered i.p. 30 minutes before the test, compound 1 produced a statistically significant CC₅₀ value compared to vehicle at all three doses of the compound.

Such data are indicative that this compound will be of therapeutic benefit.

TABLE 8
Evaluation of the effect of compound 1 in the MEST test

| | Dose | Test time post | | CC50 +/− | | % change |
|---|---|---|---|---|---|---|
| Treatment | (mg/kg) | dose (min) | N | SEM | Significance | from vehicle |
| Vehicle | — | 30 | 12 | 24.3 ± 0.4 | — | — |
| Diazepam | 2.5 | 30 | 11 | 78.5 ± 1.0 | P < 0.001 | 223% |
| Compound 1 | 2 | 30 | 12 | 30.8 ± 1.0 | P < 0.001 | 27% |
| Compound 1 | 20 | 30 | 12 | 52.5 ± 1.3 | P < 0.001 | 116% |
| Compound 1 | 200 | 30 | 12 | 197.5 ± 20.4 | P < 0.001 | 712% |

### Conclusions

These data demonstrate a therapeutic effect for compound 1. Thus, the compound may be of therapeutic value.

The compound produced a dose-related increase in MEST, suggesting that this compound exhibits anticonvulsive properties. Significant effects were observed at 2, 20 and 200 mg/kg, when compared to vehicle.

### Example 8: Preparation of Form B at 10 g Scale

Compound 1 (10.0 g) in THF (40 mL, 4 vol) was heated to 65° C. in a jacketed vessel. Ethyl acetate (30 mL, 3 vol) was added and the mixture distilled at atmospheric pressure to reduce the volume to 4 vol (jacket temperature 105° C.; distillate temperature rises from 65 to 75° C.) The process was repeated three times. Ethyl acetate (10 mL, 1 vol) was added and the mixture cooled to 20 to 25° C. over 2 hours). Heptane (40 mL, 4 vol) was added over 30 minutes and the mixture stirred for 12 hours after the addition was complete.

The mixture was filtered (P3 sinter; 3.5 cm diameter) and washed with 1:1 heptane/ethyl acetate (20 mL, 2 vol) followed by heptane (20 mL, 2 vol). The solid was dried under vacuum at 40 to 45° C. for 12 hours to give Form B material.

### References

A number of publications are cited above in order to more fully describe and disclose the invention and the state of the art to which the invention pertains. Full citations for these references are provided below. The contents of each of these references is incorporated herein.

O'Sullivan et al., “Towards Better Delivery of Cannabidiol (CBD)”, Pharmaceuticals, 2020, Vol. 13, pp. 1-15. doi: 10.3390/ph13090219

1. A crystalline form of compound 1: 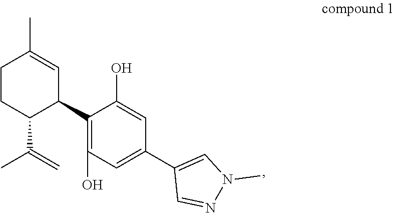 [SMILES: C=C(C)[C@@H]1CCC(C)=C[C@H]1c1c(O)cc(-c2cnn(C)c2)cc1O]
characterised by an X-ray powder diffraction (XRPD) pattern comprising a peak at 8.33 and/or 23.16±0.2 (°2θ).

2. The crystalline form of claim 1, further characterised by a thermogravimetric differential thermal analysis (TG/DTA) thermogram comprising a peak with an onset temperature (Tₒₙₛₑₜ) of 195±2° C.

3. The crystalline form of claim 1 or claim 2, further characterised by an XRPD pattern further comprising least one peak at a position (°2θ) selected from 12.61, 15.65, 16.88, 20.14, 23.62, 23.99 and 26.44±0.2.

4. The crystalline form of any preceding claim, further characterised by an XRPD pattern further comprising least five peaks at positions (°2θ) selected from 12.61, 15.65, 16.88, 20.14, 23.62, 23.99 and 26.44±0.2.

5. The crystalline form of any preceding claim, further characterised by an XRPD pattern further comprising peaks at 12.61, 15.65, 16.88, 20.14, 23.62, 23.99 and 26.44±0.2 (°2θ).

6. The crystalline form of any preceding claim, further characterised by an XRPD pattern further comprising peaks at 12.45, 13.74, 16.70, 19.91, 20.88, 22.06, 25.06 and 25.85±0.2 (°2θ).

7. The crystalline form of any preceding claim, further characterised by an XRPD pattern further comprising peaks at 13.96, 17.75, 19.73, 21.91 and 24.66±0.2 (°2θ).

8. The crystalline form of any preceding claim, characterised by an XRPD pattern substantial as shown in FIG. 6.

9. The crystalline form of any preceding claim, which is anhydrous.

10. A crystalline form of compound 1, characterised by an XRPD pattern a peak at 14.92±0.2 (°2θ).

11. The crystalline form of claim 10, further characterised by an XRPD pattern further comprising least one peak at a position (°2θ) selected from 12.91, 20.20, 24.03, 24.16 and 27.54±0.2.

12. The crystalline form of claim 10 or claim 11, further characterised by an XRPD pattern further comprising peaks at 12.91, 20.20, 24.03, 24.16 and 27.54±0.2 (°2θ).

13. The crystalline form of any of claims 10 to 12, further characterised by a TG/DTA thermogram comprising a peak with a Tₒₙₛₑₜ of 179±2° C.

14. The crystalline form of any of claims 10 to 13, further characterised by an XRPD pattern further comprising peaks at 12.21, 17.89, 19.15 and 25.12±0.2 (°2θ).

15. The crystalline form of any of claims 10 to 14, further characterised by an XRPD pattern further comprising peaks at 24.53±0.2 (°2θ).

16. The crystalline form of any of claims 10 to 15, characterised by an XRPD pattern substantial as shown in FIG. 3.

17. The crystalline form of any of claims 10 to 16, which is anhydrous.

18. A crystalline form of compound 1, characterised by an XRPD pattern comprising at least one peak at a position (°2θ) selected from 4.51, 13.86, 16.39, 18.57, 20.94, 22.25 and 25.66±0.2.

19. The crystalline form of claim 18, further characterised by an XRPD pattern further comprising peaks at 4.51, 13.86, 16.39, 18.57, 20.94, 22.25 and 25.66±0.22 (°2θ).

20. The crystalline form of claim 18 or 19, further characterised by a TG/DTA thermogram comprising a peak with a Tₒₙₛₑₜ of 76±2° C., optionally wherein the peak is associated with a weight loss of from 5 wt % to 18 wt %.

21. The crystalline form of any of claims 18 to 20, further characterised by an XRPD pattern further comprising peaks at 11.45, 17.86, 19.51 and 21.98±0.2 (°2θ).

22. The crystalline form of any of claims 18 to 21, characterised by an XRPD pattern substantially as shown in FIG. 10.

23. The crystalline form of any of claims 18 to 22, which is an ethanol solvate.

24. A crystalline form of compound 1, characterised by an XRPD pattern comprising at least one peak at a position (°2θ) selected from 9.02, 13.89, 16.41, 17.89, 18.60, 22.02, 22.29 and 25.69±0.2.

25. The crystalline form of claim 24, characterised by an XRPD pattern further comprising peaks at 9.02, 13.89, 16.41, 17.89, 18.60, 22.02, 22.29 and 25.69±0.2 (°2θ).

26. The crystalline form of claim 24 or claim 25, further characterised by an XRPD pattern further comprising peaks at 11.48 and 19.54±0.2 (°2θ).

27. The crystalline for of any of claims 24 to 26, characterised by an XRPD pattern substantially as shown in FIG. 12.

28. A composition comprising compound 1, wherein 5 wt % or more, such as 50 wt % or more, of the compound 1 is in the crystalline form of any of claims 1 to 27.

29. A composition comprising compound 1, wherein 90 wt % or more, such as 95 wt % or more, of the compound 1 is the crystalline form of any of claims 1 to 27.

30. A pharmaceutical composition comprising the crystalline form of any one of claims 1 to 27, or the composition of claim 28 or claim 29, together with one or more ingredients selected from carriers, diluents, excipients, adjuvants, fillers, buffers, binders, disintegrants, preservatives, antioxidants, lubricants, stabilisers, solubilisers, surfactants, masking agents, colouring agents, flavouring agents, and sweetening agents.

31. The pharmaceutical composition of claim 30 in a form selected from a tablet, a granule, a powder, a lozenge, a pastille, a capsule or a pill.

32. The crystalline form of any one of claims 1 to 27, or the composition of any of claims 28 to 31, for use in a method of treatment, such as in the treatment of a condition associated with seizure. 33 Use of the crystalline form of any one of claims 1 to 27, or the composition of any of claims 28 to 31, for the manufacture of a medicament, such as a medicament for the treatment of a condition associated with seizure.

34. A method of treatment comprising administering to a subject in need of treatment a therapeutically effective amount of the crystalline form of any one of claims 1 to 27, or the composition of any of claims 28 to 31.

35. The crystalline form or composition for use of claim 32, wherein the condition associated with seizure is epilepsy, generalise-onset seizure or focal-onset seizure.

36. The use for the manufacture of a medicament of claim 33, wherein the condition associated with seizure is epilepsy, generalise-onset seizure or focal-onset seizure.

37. The method of treatment of claim 34, wherein the condition associated with seizure is epilepsy, generalise-onset seizure or focal-onset seizure.

38. A method for preparing a crystalline form of compound 1, the method comprising the steps of:
a) providing a composition comprising compound 1 and a first solvent;
b) adding a second solvent; and
c) isolating the solids.

39. The method of claim 38, wherein the first solvent is selected from esters such as ethyl acetate and isopropyl acetate; ethers such as dioxane, tetrahydrofuran, 2-methyl tetrahydrofuran, methyl tert-butyl ether and cyclopentyl methyl ether; ketones such as acetone; and nitriles such as acetonitrile.

40. The method of claim 39, wherein the first solvent is ethyl acetate.

41. The method of any of claims 38 to 40, wherein step a) is carried out at from 50° C. to 80° C.

42. The method of any of claims 38 to 41, wherein the second solvent is selected from linear alkanes such as pentane, hexane, heptane and octane; cycloalkanes such as cyclopentane, cyclohexane, methylcyclohexane, cycloheptane and cyclooctane; petroleum fractions such as kerosene and petroleum ether; and aromatic hydrocarbon solvents such as benzene, toluene and xylene.

43. The method of claim 42, wherein the second solvent is heptane.

44. The method of any of claims 38 to 43, wherein step b) is carried out at from 10° C. to 30° C.

45. The method of any of claims 38 to 44, further comprising a step b-1) between steps b) and c):
b-1) holding the crystallisation mixture at a predetermined temperature for a predetermined length of time.

46. The method of claim 45, wherein the predetermined temperature is from 15° C. to 25° C.

47. The method of claim 46 or 47, wherein the predetermined length of time is from 1 hour to 24 hours.

48. A crystalline form of compound 1 prepared by the method of any of claims 38 to 47.
